(19) United States
(12) Patent Application Publication
(10) Pub. No.: US 2018/0243336 A1
(43) Pub. Date: Aug. 30, 2018

(54) ANTIMICROBIAL AND ENZYME INHIBITORY ZINC OXIDE NANOPARTICLES

(71) Applicant: THE REGENTS OF THE UNIVERSITY OF MICHIGAN, Ann Arbor, MI (US)

(72) Inventors: Jeremy Scott VANEPPS, Ann Arbor, MI (US); Nicolas A. KOTOV, Ypsilanti, MI (US)

(73) Assignee: THE REGENTS OF THE UNIVERSITY OF MICHIGAN, Ann Arbor, MI (US)

(21) Appl. No.: 15/755,394

(22) PCT Filed: Aug. 29, 2016
(86) PCT No.: PCT/US2016/049250

§ 371 Date: Feb. 26, 2018

Related U.S. Application Data
(60) Provisional application No. 62/211,509, filed on Aug. 28, 2015.

(51) Int. Cl. A61K 33/30; A61K 9/14; C01G 9/02; A61L 2/232

(52) U.S. Cl. CPC A61K 33/30; A61K 9/14; A61L 2202/24; A61L 2/232; C01P 2004/64; C01G 9/02

(57) ABSTRACT

In certain aspects, the present disclosure provides an enzyme inhibitory nanoparticle. The nanoparticle may comprise zinc oxide. The nanoparticle exhibits substantially reversible enzyme inhibition in the presence of an enzyme. In certain aspects, the shape of the nanoparticle may be a nanopyramid or a nanoplate/nanodisc. In other aspects, the present disclosure provides an antimicrobial material comprising a layer-by-layer (LBL) coating comprising a plurality of nanoparticles comprising zinc oxide. Each nanoparticle exhibits antimicrobial activity in the presence of bacteria. LBL coatings of ZnO-NP reduced Staphylococcal biofilm burden by >95%. The disclosure also provides methods of preparing an enzyme inhibitory or antimicrobial nanoparticles comprising zinc oxide.

### CROSS-REFERENCE TO RELATED APPLICATIONS

This application claims the benefit and priority of U.S. Application Ser. No. 62/211,509 filed on Aug. 28, 2015. The entire disclosure of the above application is incorporated herein by reference.

### GOVERNMENT RIGHTS

This invention is made with government support under DMR1411014, CBET1403777, and DMR1120923 awarded by National Science Foundation and W911NF-10-1-0518 awarded by the Department of Army. The government has certain rights in the invention.

### FIELD

The present disclosure relates to nanoparticles formed of zinc oxide capable of exhibiting reversible enzyme inhibition and antimicrobial effects and methods for making the same.

### BACKGROUND

This section provides background information related to the present disclosure which is not necessarily prior art.

Enzyme inhibitors are as omnipresent in living organisms as enzymes. They are relevant to a wide spectrum of clinical and technological problems: from antibacterial drugs; to treatment of diabetes, Alzheimer's disease, and some cancers, to production of foods, biofuels, and biosensors. Correspondingly, there has been considerable effort to obtain comprehensive understanding of enzyme inhibition over many years. Most studies have focused on the formation of inter-molecular lock-and-key complexes with small organic molecules or complementary proteins and peptides. However, being organic in nature these traditional enzyme inhibitors are unstable and, in turn, are degraded by other enzymes.

Given the diversity of roles that enzyme inhibitors play, it is imperative to develop new types of inhibitors with unconventional structures that circumvent degradation processes and/or generate different inhibitory effects. Some inorganic nanoparticles (NPs) have been shown to reduce enzyme activity, while others have been shown to increase activity. Furthermore, the conventional data for NP modulation of enzyme activity are limited and introduce significant uncertainty. It would be desirable to find a stable and robust inorganic nanoparticle capable of reliably and reversibly controlling or inhibiting enzyme activity.

Furthermore, despite a decade of engineering and clinical process improvements, bacterial colonization and infection remain the primary threats to implanted medical devices. The adhesion to and subsequent colonization of engineered materials by bacteria and bacterial biofilms pose significant challenges to many industries including, maritime shipping, naval engineering, waste water treatment, food safety, and healthcare. Biofilms are a particular threat to health where half of the 2 million annual healthcare-associated infections in the U.S. can be attributed to indwelling medical devices. Infected devices remain the most common cause of healthcare-associated bloodstream infection. Treatment of implant infection requires surgical extraction of a potentially precious device and/or prolonged antibiotic therapy. Surgical replacement of a device brings significant risk for serious or even life-threatening complications. Extended courses of broad spectrum antibiotics bring other complications including toxicity (e.g., renal impairment) and opportunistic infections (e.g., C. difficile colitis). Furthermore, extended antibiotic use drives the development of antibiotic resistance. Therefore, it would be desirable to have antimicrobial nanoparticles that can form robust antimicrobial materials. In certain aspects, it would be desirable to have nanoparticles that exhibit both enzyme activity inhibition and antimicrobial benefits, while being low cost and having low toxicity to mammals.

### SUMMARY

This section provides a general summary of the disclosure and is not a comprehensive disclosure of its full scope or all of its features.

In certain aspects, the present disclosure provides an enzyme inhibitory nanoparticle. The nanoparticle may comprise zinc oxide. The nanoparticle exhibits substantially reversible enzyme inhibition in the presence of an enzyme.

In another aspect, the present disclosure provides an antimicrobial material comprising a layer-by-layer coating comprising a plurality of nanoparticles comprising zinc oxide. Each nanoparticle exhibits antimicrobial activity in the presence of bacteria.

In yet another aspect, the present disclosure provides a method of preparing an enzyme inhibitory nanoparticle comprising zinc oxide. The methods may provide high levels of shape selectivity. The method may comprise reacting a precursor comprising zinc with potassium hydroxide (KOH) in the presence of an alcohol to form a zinc oxide nanoparticle. The nanoparticle has a surface comprising zinc oxide that is substantially free of capping agents, surfactants, and stabilizing agents other than the KOH.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1F. FIGS. 1A-1C are transmission electron microscopy (TEM) images of ZnO. FIG. 1A shows nanopyramids (nPYs), FIG. 1B shows nanoplates (nPLs), and FIG. 1C shows nanospheres (nSPs). FIG. 1D shows relative catalytic activity of enzyme ß-galactosidase (GAL) in the presence of three different shaped ZnO NPs (nPYs, nPLs, and nSPs) after 60 minutes incubation time. Each relative catalytic activity of GAL with ZnO NPs is normalized with respect to free enzyme activity. The initial concentration of GAL is 0.4 nM. FIG. 1E shows the values for Vₘₐₓ and FIG. 1F shows Kₘ of GAL for the same nPYs, nPLs, and nSPs, calculated from the Michael-Menten equation.

FIGS. 2A-2E. FIG. 2A shows circular dichroism spectra of GAL in the absence and presence of ZnO NPs (nanopyramids (nPYs), nanoplates (nPLs), and nanospheres (nSPs)). A molar ratio of ZnO NPs to GAL is 0.25. FIG. 2B shows gel electrophoresis of GAL with various concentrations of ZnO nanopyramids (nPYs), nanoplates (nPLs), and nanospheres (nSPs). The concentrations of ZnO NPs from column 1 to 9 are 0.00, 0.07, 0.20, 0.34, 0.48, 0.62, 0.75, 0.88, and 1.02 μM, respectively. The concentration of GAL is 360 nM. Lineweaver-Burk plots of the GAL with the various concentrations of ZnO are shown in FIG. 2C (nanopyramids), FIG. 2D (nanoplates), and FIG. 2E (nanospheres).

FIGS. 3A-3B. FIG. 3A shows a three-dimensional molecular structure and FIG. 3B shows a map of electronic potentials of GAL. Blue and red colors indicate areas with relatively positive and negative molecular potential respectively. Approximate location of the essential amino acids of the active site is highlighted with green and magenta stars.

FIGS. 4A-4F. FIGS. 4A-4C show planktonic growth curves measured turbidometrically (OD₆₀₀) for MRSA in the presence of each of the three ZnO NPs shapes (nanopyramids, nanoplates, and nanospheres) at various concentrations from 0-1.4 μM.

FIGS. 4D-4F show box plots of bacteria concentration after 10 hours of growth expressed as CFUs per ml for nanopyramids, nanoplates, and nanospheres, respectively. The limit of detection is 200 CFUs per ml. The center lines in each of FIGS. 4D-4F represent the MRSA concentration starting inoculum at time 0.

FIGS. 5A-5F. FIGS. 5A-5C are representative TEM images and FIGS. 5D-5F are selected area electron diffraction patterns of ZnO pyramids (FIGS. 5A and 5D), spheres (FIGS. 5B and 5E), and plates (FIGS. 5C and 5F) that demonstrate identical crystal lattice structures for all three shapes.

FIG. 6 shows normalized photoluminescence spectra for ZnO-NPs (nanopyramids, nanoplates, and nanospheres). PL spectra demonstrate near identical surface chemistry for each shape.

FIGS. 7A-7B. FIG. 7A shows growth curves for E. coli and K. pneumonia in the presence of increasing concentration of ZnO-NPs synthesized as pyramids, spheres, and plates. FIG. 7B shows growth curves for S. aureus, and S. epidermidis in the presence of increasing concentration of ZnO-NPs synthesized as pyramids, spheres, and plates.

FIG. 8 shows a fraction of cells that partition to an aqueous-hexadecane interface at mid-log versus stationary phase for each E. coli, K. pneumonia, S. aureus, and S. epidermidis organism.

FIGS. 9A-9C. FIGS. 9A-9C show comparisons of a dose response on the growth rate of S. epidermidis by ZnO pyramids, spheres, and plates for units of mass concentration (FIG. 9A), surface area (FIG. 9B), and particle number concentration (FIG. 9C). Data represent mean+/−standard error. Insets in FIGS. 9B and 9C are expanded views of the data at the lower end of the x-axis to delineate differences in the plates and pyramids.

FIG. 10 shows ZnO leaching measured by absorbance at 350 nm.

FIG. 11 shows box-plots of colony forming units (CFU) per square centimeter of E. coli, S. aureus, and S. epidermidis recovered from bare pegs or pegs coated in ZnO plates, pyramids, or spheres. Limits of detection for this assay are 100 CFU/cm²

FIGS. 12A-12L. Scanning electron microscopy (SEM) micrographs of bare polystyrene pegs are shown in FIGS. 12A-12C, pegs coated in ZnO spheres are shown in FIGS. 12D-12F, plates are shown in FIGS. 12G-12I, and pyramids are shown in FIGS. 12J-12L cultured with E. coli (FIGS. 12A, 12D, 12G, and 12J), S. aureus (FIGS. 12B, 12E, 12H, and 12K), and S. epidermidis (FIGS. 12C, 12F, 12I, and 12L).

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. By way of example, it may be less than 5% of the value indicated and in certain variations, optionally less than 1% of the value indicated.

Every patent publication or other literature reference discussed in the context of the present disclosure is explicitly incorporated herein by reference

In addition, disclosure of ranges includes disclosure of all values and further divided ranges within the entire range, including endpoints and sub-ranges given for the ranges. Example embodiments will now be described more fully with reference to the accompanying drawings.

The present disclosure provides an inorganic nanoparticle that is capable of use as an enzyme inhibitor or an antimicrobial agent. In certain variations, the inorganic nanoparticle comprises a metal oxide material. In certain aspects, the metal is zinc and the metal oxide is zinc oxide. The term “nano-sized” or “nano-scale” is generally less than about 1 μm (i.e., 1,000 nm). A “nano-particle” generally refers to a nano-component where all three spatial dimensions are nano-sized and less than or equal to a micrometer (e.g., less than about 1,000 nm). In accordance with the present disclosure, a nanosized particle may have at least one spatial dimension that is less than or equal to about 50 nm, optionally less than or equal to about 40 nm, optionally less than or equal to about 30 nm, optionally less than or equal to about 25 nm, optionally less than or equal to about 20 nm, optionally less than or equal to about 15 nm, optionally less than or equal to about 10 nm, optionally less than or equal to about 5 nm, and in certain variations, optionally less than or equal to about 4 nm. In certain aspects, the nanosized particle has at least one spatial dimension that is greater than or equal to about 1 nm to less than or equal to about 50 nm, optionally greater than or equal to about 1 nm to less than or equal to about 25 nm, optionally greater than or equal to about 1 nm to less than or equal to about 20 nm, optionally greater than or equal to about 1 nm to less than or equal to about 15 nm, optionally greater than or equal to about 1 nm to less than or equal to about 10 nm, and in certain variations, optionally greater than or equal to about 1 nm to less than or equal to about 5 nm.

In certain other aspects of the present teachings, a nanoparticle has all three spatial dimensions that are less than or equal to about 50 nm, optionally less than or equal to about 40 nm, optionally less than or equal to about 30 nm, optionally less than or equal to about 25 nm, optionally less than or equal to about 20 nm, optionally less than or equal to about 15 nm, optionally less than or equal to about 10 nm, optionally less than or equal to about 5 nm, and in certain variations, all of the spatial dimensions are less than or equal to about 4 nm. In certain other aspects, all dimensions of the nanosized particle are greater than or equal to about 1 nm to less than or equal to about 50 nm, optionally greater than or equal to about 1 nm to less than or equal to about 25 nm, optionally greater than or equal to about 1 nm to less than or equal to about 20 nm, optionally greater than or equal to about 1 nm to less than or equal to about 15 nm, optionally greater than or equal to about 1 nm to less than or equal to about 10 nm, and in certain variations, optionally greater than or equal to about 1 nm to less than or equal to about 5 nm.

Zinc oxide nanoparticles (ZnO NPs) have been synthesized in accordance with certain aspects of the present disclosure that have an average particle size of less than 20 nm. The zinc oxide nanoparticles are formed with specific different shapes (e.g., spheres, plates, and hexagonal pyramids) without the use of traditional capping agents, stabilizers, or surfactants. Such traditional capping agents, stabilizers, and surfactants have in the past been required to form zinc oxide nanoparticles. The synthesis methods provided by the present disclosure generate particles with nearly identical surface chemistry, but vastly different and controllable shapes. As such, zinc oxide nanoparticles have been formed that can act as shape-dependent biomimetic enzyme inhibitors. The shape-dependence provides a new and enhanced level of engineering control over the inhibitory function of the zinc oxide nanoparticles. While nanoparticles have previously been used to inhibit enzymes, it has been the result of irreversible binding and/or denaturation of the enzyme itself. However, the nanoparticles provided by certain aspects of the present teachings are unique in that the interaction with the enzyme is shape-dependent, reversible, and furthermore does not result in enzyme denaturation. This more closely resembles the interaction of enzymes with biological inhibitors in nature, thus providing biomimetic enzyme inhibition.

While in certain preferred variations, the inorganic nanoparticle comprises zinc oxide, similar shape effects can be observed for inorganic nanoparticles formed from other materials besides zinc oxide. Such other materials include, but are not limited to, zinc sulfide, zinc telluride, zinc selenide, cadmium chalcogenides, manganese oxide, silica oxide, alumina oxide aluminosilicate, metal oxides, carbon nanomaterials, and other metals.

In various aspects, the present disclosure contemplates a platform for the engineering of inorganic nanoparticles as biomimetic enzyme inhibitors. The benefits of such engineered particles over traditional enzyme inhibitors are multi-fold. The inorganic metal oxide (e.g., zinc oxide) is resistant to normal biological degradation processes giving them a long life span. The size and shape of the nanoparticle can be controlled to tune the level or extent of enzyme inhibition. Zinc oxide has been demonstrated to be safe and is used in many sunscreens and other topical skin and personal care products. Zinc oxide has antimicrobial properties. The enzyme inhibition, which is nonspecific, provides antimicrobial activity against multiple antibacterial targets which reduce the potential for the development of tolerance or resistance. Furthermore, the nanoparticles can easily be applied to surfaces or substrates via certain layer-by-layer methods according to the present teachings to create bioactive surface coatings.

As such, the nanoparticle provided by certain aspects of the present disclosure can be used as an antimicrobial particle that forms antimicrobial materials that can be used in a variety of applications, including for in-dwelling implanted medical devices, sprays/wipes for disinfecting medical equipment, bedding, or other healthcare devices, by way of non-limiting example, without the toxic effects of current disinfectants. By antimicrobial, it is meant that the material inhibits or prevents growth of microbes, including bacteria, fungi, viruses, and other spore forming organisms. As noted above, such antimicrobial activity is believed to be related to the nanoparticle's ability to inhibit enzyme activity. In certain variations, an antimicrobial material according to the present disclosure exhibits antimicrobial activity. The antimicrobial activity and effects follow the same shape-dependent patterns of the enzyme inhibition effects for the nanoparticles.

By way of example, the antimicrobial material may exhibit an antimicrobial activity in the presence of bacteria. For example, the antimicrobial zinc oxide nanoparticles prepared in accordance with certain aspects of the present disclosure may reduce a biofilm burden (e.g., a biofilm of Gram-positive bacteria like Staphylococcal) by greater than or equal to about 75% as compared to a surface of a substrate without any antimicrobial material (comprising zinc oxide nanoparticles), optionally greater than or equal to about 80%, optionally greater than or equal to about 85%, optionally greater than or equal to about 90%, and in certain variations, optionally greater than or equal to about 95% of the biofilm burden on the substrate as compared to a substrate without any antimicrobial material.

In certain aspects, the nanoparticles provided by the present teachings define a shape selected from the group consisting of: polyhedrons, pyramids, cones, discs or plates, rods, cylinders, stars, spheres, rectangles, and combinations thereof. Examples of pyramids may include triangular pyramids, square pyramids, pentagonal pyramids, hexagonal pyramids, heptagonal pyramids, octagonal pyramids, and the like. In certain aspects, the nanoparticles provided by the present teachings define a non-spherical shape selected from the group consisting of: polyhedrons, pyramids, cones, discs or plates, rods, cylinders, stars, rectangles, and combinations thereof. Examples of pyramids may include triangular pyramids, square pyramids, pentagonal pyramids, hexagonal pyramids, heptagonal pyramids, octagonal pyramids, and the like. In certain aspects, each nanoparticle has a non-spherical shape selected from the group consisting of: pyramids, cones, discs, plates, and combinations thereof. In certain preferred aspects, the nanoparticle has a pyramidal shape.

In certain aspects, the nanoparticle defines a non-spherical shape. In other aspects, the shape may have an aspect ratio of greater than or equal to about 1.25. In certain aspects, the shape has the presence of apexes, edges and other geometrical features that can facilitate geometrical match with other nanoscale particles, biological molecules with nanoscale dimensions, and/or globular polymers. Spheres or spheroid type shapes typically have aspect ratios of about 1. Thus, in certain aspects, a nanoparticle has a shape and therefore a relatively high aspect ratio (AR) (defined as the longest dimension divided by a second dimension (e.g., diameter) of the component that is orthogonal to the longest dimension) of greater than or equal to about 1.25 up to about 7.

In certain variations, the nanoparticle comprising a zinc oxide material exhibits substantially reversible enzyme inhibition in the presence of an enzyme. By “enzyme inhibition” it is meant that when a plurality of the nanoparticles is combined with an enzyme, the enzyme's activity (reaction rates) are reduced by greater than or equal to about 50% as compared to the enzyme's activity in the absence of the nanoparticles, optionally reduced by greater than or equal to about 60%, optionally reduced by greater than or equal to about 70%, optionally reduced by greater than or equal to about 75%, optionally reduced by greater than or equal to about 80%, optionally reduced by greater than or equal to about 85%, optionally reduced by greater than or equal to about 90%, optionally reduced by greater than or equal to about 95%, optionally reduced by greater than or equal to about 97%, optionally reduced by greater than or equal to about 98%, and in certain variations optionally reduced by greater than or equal to about 99% as compared to an enzyme activity in the absence of the nanoparticles.

By “substantially reversible” enzyme inhibition it is meant that when the nanoparticles are removed from the environment in which the enzyme is present, that the subsequent enzyme activity levels are greater than or equal to about 70% of an initial enzyme activity level prior to exposure to the inhibitory nanoparticles, optionally subsequent enzyme activity is restored to greater than or equal to about 80%, optionally greater than or equal to about 85%, optionally greater than or equal to about 90%, optionally greater than or equal to about 95%, optionally greater than or equal to about 97%, optionally greater than or equal to about 98%, and in certain variations optionally greater than or equal to about 99% of the initial enzyme activity.

Non-limiting examples of suitable enzymes that may have activity include hydrolases, proteases, amylases, lipases, cellulases, laccases, metalloproteinases, oxidases, carboxylases, ligases, urease, uricase, creatininase, esterases, pectinases, hydroxylases, catalases, acylase, catalase, esterase, and any combinations or equivalents thereof. In certain aspects, the enzyme may be a typical enzyme ß-galactosidase (GAL) or peroxidase enzymes.

Enzyme inhibitors are bioactive molecules, ubiquitous in all living systems, whose inhibitory activity is strongly dependent on their molecular shape. In the present teachings, it is shown that small zinc oxide nanoparticles—for example, pyramids and plates—possess the ability to inhibit a typical enzyme ß-galactosidase (GAL) activity in a biomimetic fashion. Enzyme inhibition by the ZnO NPs prepared in accordance with the present disclosure is reversible and follows classical Michaelis-Menten kinetics with parameters strongly dependent on their geometry. Association of GAL with specific ZnO NP geometries interferes with conformational reorganization of the enzyme necessary for its catalytic activity. The strongest inhibition is observed for ZnO nanopyramids and compares favorably to that of the best natural GAL inhibitors, while having the additional benefit of being resistant to proteases. Besides the fundamental significance of this biomimetic behavior of NPs prepared in accordance with the present disclosure, their capacity to serve as degradation-resistant enzyme inhibitors is technologically attractive and is substantiated by strong shape-specific antibacterial activity against methicillin-resistant Staphylococcus aureus (MRSA) endemic for most hospitals in the world.

Nanoscale dimensions and surface chemistries of conventional nanoparticles (NPs) coated with organic surface moieties are similar to those of many protein enzyme inhibitors. NPs can potentially provide a biomimetic platform to control the catalytic activity of enzymes by replicating the non-covalent interactions between enzymes and traditional biological inhibitors. Improved specificity of inhibition can be achieved by controlling the shape, as well as the surface chemistry, of NPs. Shape effects are an important design parameter, because a large variety of NPs with very diverse geometries can now be prepared. Reversibility of NP-enzyme binding in conventional NPs can be realized via better control of electrostatic and vdW forces.

The present disclosure provides that NP inhibition of biocatalytic processes strongly depends on their shape, which may vary from very weak to exceptionally strong inhibition without any denaturation of the enzyme. Analysis of the enzyme inhibition kinetics using Michaelis-Menten formalism reveals inhibitor properties and mechanisms not previously seen for other inorganic NPs. Rather, the nanoparticles provided by the present teachings mimic the properties and mechanism of traditional small molecule-, DNA-, and protein-based inhibitors. Furthermore, the strong, shape-dependent inhibitory activity is also observed for planktonic growth of methicillin-resistant Staphylococcus aureus (MRSA).

The NP inhibitors of the present disclosure are desirably variable in shape, biocompatible and desirably inexpensive. The NPs are also small in size and made from light elements in order to reduce van der Waals (vdW) forces. Balancing non-specific vdW attraction with other interactions minimizes denaturation of proteins on the NP surface and NP agglomeration in dispersion. Both factors impede the accuracy of the kinetics analysis, mechanism of inhibitory activity, and the practicality of such inhibitors.

Therefore, the present disclosure provides in certain aspects nanoparticles formed from ZnO NPs having an average diameter of less than 20 nm. The effect of such ZnO NPs on the activity of ß-galactosidase (GAL), whose structure and enzymatic activity has been extensively characterized, is investigated herein. GAL is a representative carbohydrate energy metabolism enzyme in biological systems. GAL is a hydrolase that hydrolyses beta-galactosides into monosaccharides.

In certain aspects, the present disclosure provides methods of forming nanoparticles comprising zinc oxide having a controlled shape. The methods form a plurality of nanoparticles comprising zinc oxide that have a high shape selectivity. In certain variations, a method of preparing an enzyme inhibitory nanoparticle comprising zinc oxide is provided. The method may comprise reacting a precursor comprising zinc with potassium hydroxide in the presence of an alcohol to form a zinc oxide nanoparticle. The nanoparticle has a surface comprising zinc oxide, but that is substantially free of capping agents, surfactants, and stabilizing agents other than the KOH. Nanoparticles with surface carrying specific organic functional groups can be prepared when organic molecules with such functional groups are present around nanoparticles during the reaction. Atoms of nanoparticles located in apexes and edges have higher reactivity than those located in the middle of the crystal plane. The molecular geometry of the nanoparticles and its complementarity with biomolecules, other nanoscale particles, globular polymers, and the like can be varied; it can acquire different degrees of asymmetry and for different reaction condition (temperature, pH, concentration, and the like).

In certain aspects, such a shape is one of those previously discussed above. In certain aspects, the zinc oxide nanoparticle has a shape selected from the group consisting of: pyramids, discs or plates, and spheres. For example, a precursor comprising zinc may be zinc acetate hydrate (Zn(Ac)₂.2H₂O) that can be dissolved in an anhydrous alcohol, such as methanol (MeOH). Other suitable precursors besides zinc acetate may include other soluble salts of zinc and other metals separately or in combination of zinc acetate. The dissolved zinc acetate may be heated and refluxed, for example, for an hour. Control of the shape of the particles depends upon the conditions for adding potassium hydroxide (KOH). In one variation that forms nanoplates, potassium hydroxide (KOH) can be added and dissolved in deionized water (or another aqueous solution). The dissolved KOH may be heated and refluxed, for example, for 14 hours. Forming nanospheres is a similar process, but KOH is dissolved in anhydrous alcohol (e.g., methanol) instead of deionized water. Nanopyramids are synthesized by first mixing KOH with the zinc acetate hydrate, before adding anhydrous methanol and refluxing for approximately 48 hours. Precipitates of the nanoparticles are collected and then washed.

Therefore, the various shapes are prepared using similar reactions without the use of surfactants or capping agents, aside from the presence of KOH, in order to minimize the effect of different surface chemistry and surface distribution of those molecules on the interaction with the bacterial cell surface. Besides the examples of platelets, hexagonal pyramids with a shape that allows “docking” of some complementarity to the geometry of the biological molecule, their nanoscale species, and globular proteins, also can be formed and used.

The nanoparticles formed share high crystallinity and nearly identical surface chemistry, differing only in shape and size. In certain aspects, the nanoparticle formed has a surface comprising zinc oxide, but is substantially free of capping agents, surfactants, and stabilizing agents other than potassium hydroxide (KOH). By “substantially free” it is meant that the surface does not contain any intentionally added surfactants, capping agents, or stabilizing agents other than KOH during the reaction process, although there may be negligible levels of impurities present, for example, the surface comprises less than or equal to about 0.1% of any surfactants, capping agents, or stabilizing agents aside from KOH.

### Example 1

Materials

ß-Galactosidase from Escherichia coli (GAL) and resorufin ß-D-galactopyranoside are purchased from Sigma and used without further purification. Zn acetate dihydrate, Zn(CH₃COO)₂.2H₂O, is purchased from Aldrich. Buffer solution (pH 7.5) and tetrabutylammonium bromide are obtained from Fluka. Sodium resorufin are obtained from Molecular Probes, Invitrogen.

Synthesis of ZnO NPs

The shape of ZnO nanoparticles is varied to obtain hexagonal nanopyramids, nanoplates, and nanospheres. The NPs are prepared using similar reactions like those discussed previously above, without stabilizers to minimize the effect of the different surface chemistry and distribution of stabilizers on the intermolecular interactions with enzymes.

In a typical method of the preparation of nanoplates, 2.75 g of Zn(CH₃COO)₂.2H₂O in 150 ml of ethanol is heated to reflux with stirring for 1 hour, and then 0.5 g of KOH dissolved in 5 ml of deionized water is added. After 12 hours stirring, the white precipitates are purified by washing several times with methanol.

The nanospheres are prepared by the same technique as the nanoplates, but by using 0.5 g of KOH dissolved in 5 ml ethanol, instead of deionized water.

Nano pyramids are synthesized by first mixing 0.2 g KOH with the 5.5 g Zn(Ac)₂.2H₂O, before adding anhydrous methanol and refluxing for 48 hours.

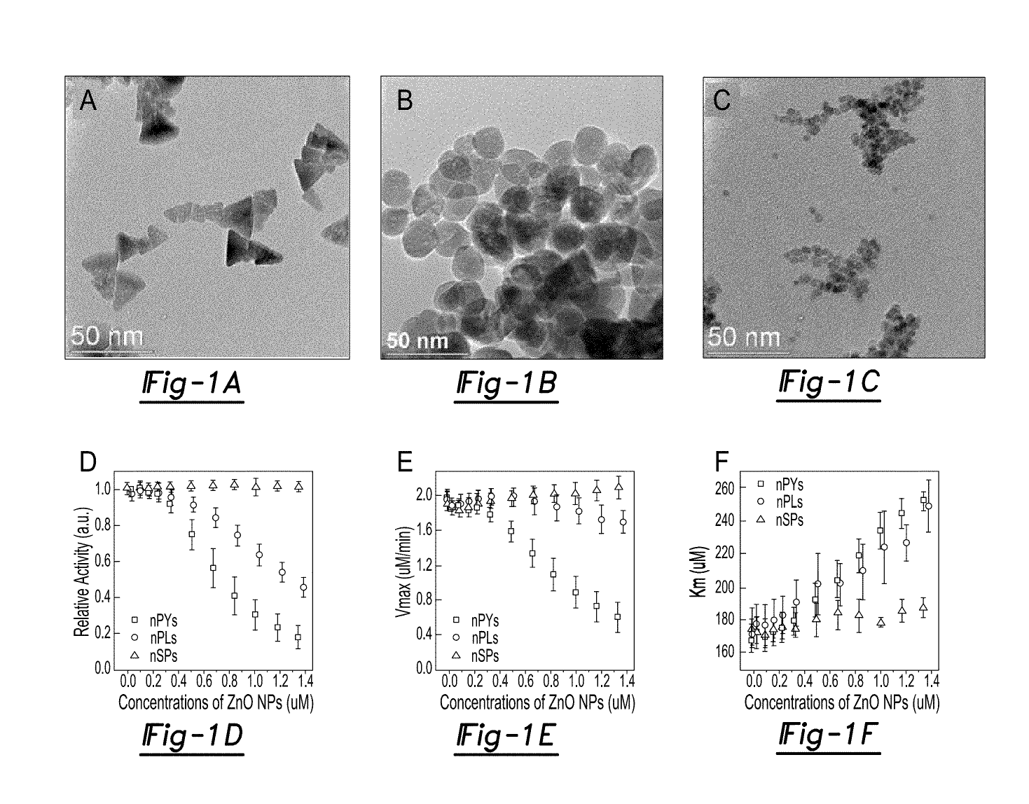

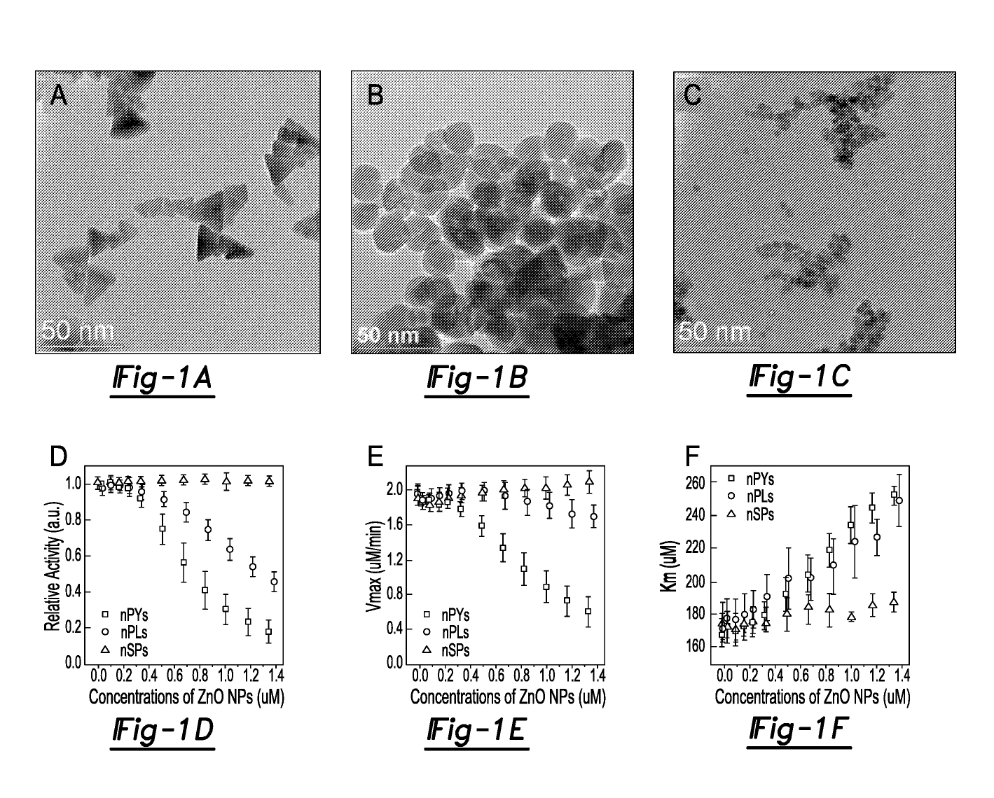

The edges of the hexagonal base of nanopyramids are measured to be about 15 nm on average, while their side edges are about 18 nm (FIG. 1A). The diameter and thickness of nanoplates are 18.4±2.9 nm and 3.5±0.2 nm, respectively (FIG. 1B). The diameter of nanospheres is 4.4±0.5 nm (FIG. 1C).

Catalytic Activity Measurement of ß-Galactosidase

The catalytic activity of GAL is determined by the increase of fluorescence intensity with time due to the accumulation of resorufin which is formed by hydrolysis of resorufin ß-D-galactopyranoside (RGP) via GAL.

The concentration of GAL is kept constant at 0.4 nM while eight different concentrations of resorufin ß-D-galactopyranoside within the range of 20-300 μM are applied. Before the measurement of the catalytic activity, GAL is incubated with varying concentrations of ZnO NPs in 20 mM sodium phosphate buffer solution (pH 7.5) for 1 hour at room temperature with gentle mixing. The catalytic reactions are started by addition of 50 μL of resorufin ß-D-galactopyranoside into 100 μL of the mixture of ZnO NPs and GAL. The fluorescence intensities are observed using fluorescence microplate reader from BioTek every minute in order to determine the values time profiles of product formation and the initial reaction rate (V₀), at each concentration of ZnO NPs. The initial linear phase lasted approximately 5 min. The intensity of fluorescence is converted into concentrations of the product (resorufin) using a fluorescence standard curve.

Considering the observations by Wang et al., “Soft Interactions at Nanoparticles Alter Protein Function and Conformation in a Size Dependent Manner,” Nano Lett. 11, pp. 4985-4991 (2011), the smaller ZnO spherical particles might be expected to have the strongest inhibition effects. However, experimental findings here are contrary to initial expectations. The inhibition efficiency greatly increased from nanospheres to nanoplates to nanopyramids (FIG. 1D). Continuous decrease of enzyme activity is observed with increasing concentrations of nanopyramids and nanoplates, while the enzyme activity is found to be virtually invariant for all concentrations of nanospheres. For the latter, inhibition of GAL is not observed even when the concentration of nanospheres exceeded 1.2 μM. For any concentration, nanopyramids show much higher inhibitory effect on GAL than nanoplates. For example, with 0.5 μM concentration of nanopyramids, the activity of GAL is reduced by approximately 25% of the original, whereas the similar concentration of nanoplates led to only approximately 9% enzyme activity loss. When the concentration of nanopyramids increased to approximately 1.2 μM, the activity of GAL dropped to approximately 20% of the original. However, in the case of nanoplates, GAL still retained about 50% activity.

The preliminary explanation for the mechanism of inhibition involved the denaturation of enzymes on NP surface and/or charge effects, as was observed previously. However, the experimental data here suggested against these mechanisms for the case of GAL and ZnO.

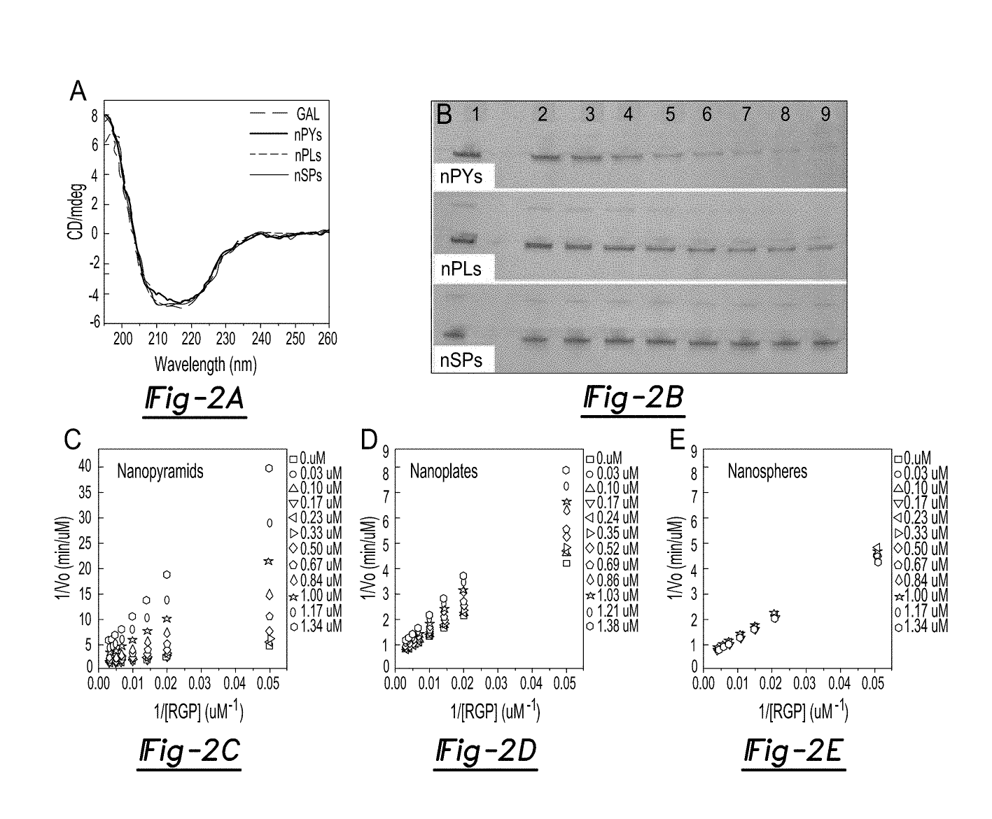

Circular dichroism spectra indicate that the conformation of GAL did not change appreciably in the presence of any concentration or shape of ZnO NPs (FIG. 2A). Circular dichroism (CD) spectra are obtained using Aviv model 202 spectrometer. For each sample, five CD spectra are recorded and averaged. Then the spectra are smoothed using Adjacent-Averaging method. UV-vis spectroscopy is carried out on an 8453 UV-vis ChemStation spectrophotometer produced by Agilent Technologies. A quartz cuvette with an optical path length of 1 cm is used for both CD and UV-vis measurements. The transmission electron microscopy (TEM) images are obtained using a JEOL 2010F analytical electron microscope at 200 keV. Atomic force microscopy (AFM) is performed with a digital Instruments NanoScope IIIa surface probe microscope. AFM images are analyzed using NanoScope R III software. The inhibition is found to be reversible, unlike cases of NP inhibition previously observed. GAL activity is completely restored when NPs are removed by ethylenediaminetetraacetic acid (EDTA).

Electrostatic attraction is another possible explanation for the inhibition and its dependence on NP shape; this mechanism would follow the model considered for gold NPs coated with monolayers of charge-bearing thiols and imply complete or partial denaturation accompanied by the conformational change. The isoelectric points of GAL and ZnO NPs are pH 4.6 and pH 9, respectively. For intermediate pH 7.5, they are oppositely charged. The zeta potentials measurements are performed by Zetasizer Nano ZS from Malvern Instruments. The electrokinetic zeta potential, ζ, is almost identical for all ZnO NPs used here and therefore, the electrostatic attraction between GAL and ZnO NPs is expected to be nearly the same regardless of shape. This indicates that the inhibition mechanism observed between ZnO and GAL is different from what was observed before with gold or silica NPs of different sizes.

Enzyme Kinetics and Calculation of Kᵢ

Inability of the previous models to adequately explain the trends of GAL inhibition by ZnO of different shapes prompted evaluation of the inhibition mechanism of ZnO NPs in greater detail. Following the classical Michaelis-Menten theory of enzyme kinetics, a typical enzyme reaction is described as:

$$E + S {⇄}_{{k}_{{—}^{1}}}^{{k}_{1}} [ ES ] {⇄}_{{k}_{{—}^{2}}}^{{k}_{2}} E + P \qquad ( 1 )$$

For the hydrolysis reaction in this example, the substrate (S) is non-fluorescent RGP. GAL enzyme (E) hydrolyzes RGP via an intermediate enzyme-substrate complex (ES) then dissociates releasing the fluorescent product resorufin (P). Taking advantage of its strong fluorescence, the kinetics of product accumulation is investigated for various concentrations of S, E, and ZnO NPs. The four rate constants in Eq. 1 (k₁, k₂, k₋₁, and k₋₂) are determined and found to be independent of the concentrations and shapes of ZnO NP as well as concentrations of S and E. This point is significant because it further affirms the conclusion that primary, secondary, and tertiary structure of GAL is intact. If the chemical structure or conformation were not retained when ZnO interacts with GAL, there should have been marked changes in some or all kinetic constants. Therefore, the possibility can be excluded that ZnO NPs might induce “mutation” of the enzyme by substituting one of the catalytic nucleophiles involved in the binding of substrate.

The calculation of enzyme kinetics parameters followed the cannons of enzymatic catalysis represented by the reaction (1). Kinetic constants in this equation are calculated following the protocol as described. Briefly, time-dependent profiles of P are found to fit very well to a two-exponential function, I(t)=A₁exp(−t/τ₁)+A₂exp(−t/τ₂), where τ₁ and τ₂ are the two characteristic reaction times. Using the plots of τ1⁻¹+τ2⁻¹ and τ1⁻¹·τ2⁻¹ versus the concentrations of GAL and RGP, the four rate constants in Eq. 1 could be determined. All rate constants are specific to the enzyme-substrate pair.

The Michaelis-Menten parameters Kₘ and Vₘₐₓ for GAL with ZnO NPs, are evaluated using Lineweaver-Burk analysis, a linear transformation of the Michaelis-Menten equation and Vₒ. The nanoplates demonstrated a competitive inhibition mechanism. For this mechanism type, the Kᵢ is determined from the slope of the Km vs [I] plot as:

$$Slope = \frac{{K}_{m}}{{K}_{i}} \qquad ( 2 )$$

where [I] in this case is the concentration of ZnO nanoplates (FIG. 1F). For the mixed inhibition mechanism demonstrated by nanopyramids, a modified form of the classical Michaelis-Menten equation is used and applied to obtain the Kᵢ values for ZnO nPYs.

$$\frac{1}{{V}_{m}} = \frac{{K}_{m}}{{V}_{m}} ( 1 + \frac{[ I ]}{{K}_{i}} ) \cdot \frac{1}{[ S ]} + \frac{1}{{V}_{m}} ( 1 + \frac{[ I ]}{α {K}_{i}} ) \qquad ( 3 )$$

To determine these parameters, the specific Lineweaver Burke plot for nanopyramids shown in FIG. 2C is translated. Specifically, the y-intercepts vs [I] are plotted and the slopes versus [I] are also plotted, where [I] in this case is the concentration of ZnO nanopyramids. The x-intercept of the y-intercept versus [I] plot is equal to −αKᵢ, while the x-intercept of the slope vs [I] plot is equal to −Kᵢ. Typical values of Kᵢ for GAL inhibitors found in nature range from 2 μM to 220 mM. The lowest reported value of and engineered GAL inhibitor Kᵢ is 0.11 nM.

ZnO NPs thus appear to be acting with respect to GAL largely as traditional inhibitors. Hence, the NP induced inhibition is determined not by the change of reaction kinetics (e.g., intrinsic rate constants k₁, k₂, k₋₁, and k₋₂) as was the case in previous studies, but by the relative binding between enzyme, substrate, and inhibitor. This conclusion also stipulates that one can apply traditional enzyme inhibitor formalisms to describe NP inhibition of GAL. When an inhibitor binds exclusively to the free enzyme and the conversion of substrate is prevented in such complex, the mechanism is labeled competitive inhibition. If the inhibitor binds only to enzyme-substrate complex, the inhibition is called uncompetitive. When the inhibitor could bind both to the free enzyme and intermediate complex, it is denoted as a noncompetitive (mixed) mechanism. The inhibition kinetics and mechanism for GAL by different ZnO NPs can be analyzed using the Michaelis-Menten equation, Vₒ=[Vₘₐₓ·S/(Kₘ+S)], where Vₒ is initial rate of the enzyme reaction, S is the concentration of substrate, Vₘₐₓ is the maximum reaction rate when E exists primarily as complex with substrate, ES. Kₘ is the Michaelis constant which gives the numerical value of the substrate concentration when reaction rate is equal to half of Vₘₐₓ; it describes the affinity of the enzyme for the substrate. The three inhibition mechanisms can be distinguished by the difference in trends in Vₘₐₓ and Kₘ expressed as plots relating the initial rates and substrate concentration also known as Lineweaver-Burk analysis.

Using this conceptual framework of traditional biomolecular inhibitors to describe the inhibitory effects of NPs, nanospheres had little effect on the enzymatic activity of GAL and resulting Lineweaver-Burk plot (FIG. 2E); Kₘ of GAL in presence of nanospheres remained unchanged at 178±5.5 μM. Note that this value of Kₘ is typical for galactosidase-RGP pair. Likewise, nanospheres had very little effect on Vₘₐₓ of GAL (FIG. 1E). In the case of nanoplates, Kₘ increases as the concentration of nanoplates increased (FIG. 1F) while Vₘₐₓ remained unchanged (FIG. 1E). For nanopyramids, consistent monotonic increase in Kₘ is observed with concomitant reduction of Vₘₐₓ for GAL as the concentration of nanopyramids increases (FIGS. 1E and 1F). These trends of change for both Kₘ and Vₘₐₓ of GAL in the presence of ZnO nanopyramids can also confirmed by another graphical representation of enzyme kinetics, an Eadie-Hofstee plot. The values of both y- and x-intercepts, indicated by Vₘₐₓ and Vₘₐₓ/Kₘ respectively, decrease with increasing the concentrations of ZnO nanopyramids. So, the binding affinity of substrate, i.e., RGP to GAL, is reduced by both nanopyramids and nanoplates. However, there is no significant difference between Kₘ values for the same concentrations of nanopyramids and nanoplates, implying that the relative effect of nanopyramids and nanoplates on the binding of RGP to GAL is similar.

Translating this data to Lineweaver-Burk analysis allowed for relative comparison of the differential effect of shape and delineated the specific inhibition mechanism. For nanoplates, the slopes in the Lineweaver-Burk plots (FIGS. 2C-2E) increased with increasing the concentrations of nanoplates while the y-intercepts are almost unchanged (FIG. 2D). In terms of the Michaelis-Menten description of inhibition kinetics, the gradual increase in Kₘ and relatively unchanged Vₘₐₓ for nanoplates match the competitive inhibition mechanism. The Kᵢ for this competitive inhibition behavior was =3 μM.

In the case of nanopyramids, both the slopes and y-intercepts increased with increasing concentration of NPs (FIG. 2C). Such trends are not frequent among traditional inhibitors and correspond to noncompetitive or mixed inhibition behavior. That is, both the ability of the substrate to bind to the reactive center and the enzyme's ability to carry out the catalytic reaction are reduced. Combination of these effects yields a synergistic increase in the inhibitory activity. Using the Lineweaver-Burk data, the competitive binding constant between the enzyme and the inhibitor was calculated to be Kᵢ=0.72 μM, whereas the binding constant for the inhibitor and enzyme-substrate complex (representing the noncompetitive component leading to dependence of Vₘₐₓ on the inhibitor concentration) was calculated to be αKᵢ=1.39 μM. The inhibitor binds more readily to the free enzyme than the enzyme-substrate complex, but only by a factor of two. Given that these NPs are not true substrate or transition state analogs in chemical structure, this is not entirely surprising. The values of Kᵢ and αKᵢ place nanopyramids among the best known natural inhibitors for GAL enzyme.

Looking into greater details of the inhibitory activity, the decrease of Vₘₐₓ in the presence of nanopyramids suggests that the substrate concentration has no influence on the degree of enzyme inhibition and the inhibition ability of nanopyramids is preserved even at high concentrations of RGP. That is, RGP cannot outcompete the ZnO nanopyramids, which leads to high inhibitory activity. Considering that the maximum reaction rate according to Eq. 1 is defined as Vₘₐₓ=k₂≮Eₒ, where Eₒ is the total concentration of enzyme and k₂ characterizes how fast ES converts into P, the decrease of Vₘₐₓ in the presence of nanopyramids should originate from the concentration decrease of Eₒ due to the overall constant value for k₂. Because the measurements of enzyme activity are conducted with the same initial concentration of GAL, the decrease of Eₒ originates from a decrease in the relative number of GAL molecules displaying catalytic activity with increasing concentrations of nanopyramids.

Because there are no denaturation-related structural changes in GAL upon interactions with NPs as indicated by UV-vis and circular dichroism (CD) (FIG. 2A) spectra, a more specific shape-dependent binding between ZnO NPs and GAL attributed to non-covalent and non-electrostatic forces is believed to be responsible for the inhibition. Indeed, electrophoretic mobility assays between GAL and the various ZnO NP shapes indicate that GAL was bound more tightly to nanopyramids than nanoplates and significantly more so than to nanospheres (FIG. 2B). The trend here repeats the trend in inhibition activity in FIG. 1D. Note that GAL-NP binding may not be associated with a specific stoichiometry as is the case of many traditional inhibitors that originate with the combination of specific and non-specific binding between the enzyme and inhibitor.

Gel Electrophoresis

Binding of GAL with ZnO NPs is determined using electrophoretic mobility assay using precast polyacrylamide gels. Electrophoresis runs are performed in a mini-PROTEAN Tetra cell with a constant voltage of 130V for 1 hour, and gels are placed in Coomassie stain solution. All reagents and equipment are from Bio-Rad. The patterns of the observed bands indicate that the mobility of free enzyme gradually decreases with increasing concentrations of nanopyramids. The same is observed for nanoplates, but to a lesser extent. For a given concentration of ZnO NPs, the mobilities of GAL with nanopyramids are always lower than one with nanoplates. Nanospheres had no effect on the electrophoretic mobility of the enzyme.

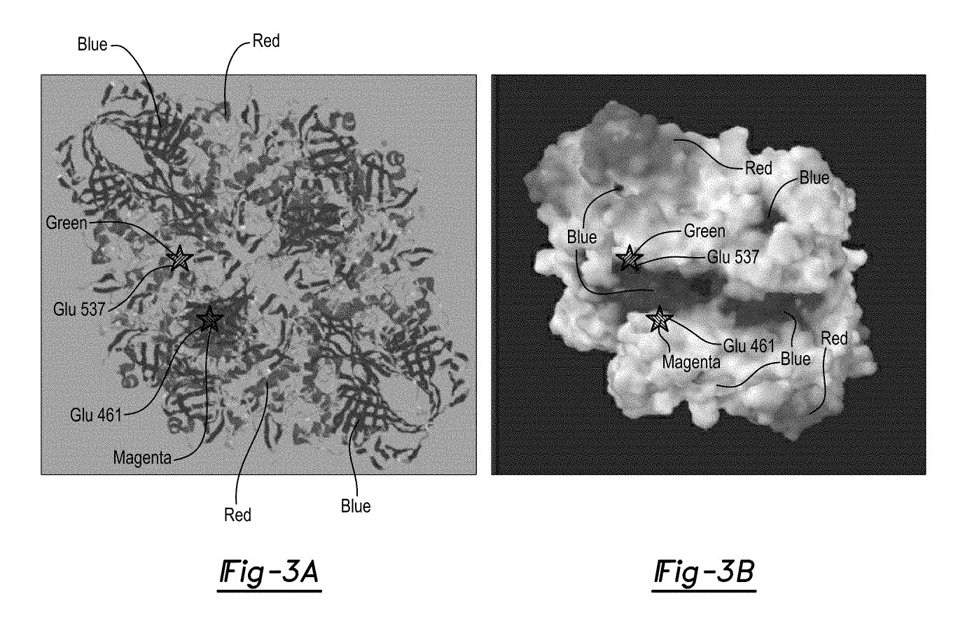

To explain why the inhibitory activity and binding between GAL and ZnO NPs are dependent on the shape, it is instructive to correlate the kinetic data with the location of the active site on the molecular structure of the enzyme. The functional form of GAL is known to be a tetramer comprised of four identical subunits (FIG. 3A). There is a continuous network of grooves running along the GAL surface (FIG. 3B). The four active sites are located at the bottom of such surface grooves and correspond to residues 335-624 in each of the monomers forming the tetrameric barrel protein. The first step in the molecular operations of the active site is the formation of a covalent bond between galactose and Glu 537 (indicated by a green star in FIGS. 3A-3B) initiated by proton donation from Glu 461 (indicated by a magenta star in FIGS. 3A-3B). The second step is the displacement of the substrate with water initiated by proton abstraction by Glu 461. The distance between Glu 537 and Glu 461 is ca 3.5 nm while the molecular size of galactose is 0.6 nm. Therefore, the active site requires substantial geometrical transformation during the reaction.

It is believed that the inhibitory action of the NPs prepared in accordance with certain aspects of the present disclosure is related to their interference with the molecular mobility of the reactive center facilitated by site-specific electrostatic attraction to the domain surrounding it (FIG. 3B). The interplay of the complex electrostatic interactions determined by the potential map (FIG. 3B), hydrogen bonds, van der Waals interactions, and the shapes of the protein and the NPs results in the strong shape dependence of the inhibitory activity of NPs on their geometry. One depiction of such a shape effect can be the ability of nanoplates and nanopyramids to partially penetrate into the grooves where the active center is located and interfere with its reconfiguration needed for the catalytic reaction. Greater inhibitory activity of nanopyramids compared to nanoplates is related to better geometrical match with the enzyme surface due to sharper apexes and edges. Enhanced inhibitory activity compared with traditional inhibitors is related to the fact that the relatively small molecules of substrate have particular difficulty displacing the heavy NPs.

The mode of NP-enzyme interaction described above is unlikely to lead to high specificity of inhibition. Nevertheless, it can play a considerable role in the biology. Moreover, the ability to interact with multiple structurally similar enzymes and enhanced resilience against biodegradation characteristic for inorganic materials can be of great advantage for many applications exemplified here by the antibacterial activity of ZnO NPs. ZnO NPs are known to have a broad spectrum of antibacterial action which has often been associated with the generation of reactive oxygen species (ROS) or disruption of bacterial cell wall. However these hypotheses cannot explain antibacterial action of ZnO in its entirety, for instance the high antibacterial activity in the absence of light and the increased efficacy with reduction in particle size. Inhibition of a family of enzymes represented by GAL leading to global dysfunction of the organism could also be a mechanism of the antibacterial properties of ZnO NPs. It is also known that many enzymes responsible for ROS scavenging require divalent cofactors like GAL.

If inhibition of GAL-like enzymes contributes to the antibacterial action, one would expect a similar pattern of shape-specific inhibition of bacterial growth as was seen for GAL. Conventional expectations based on other mentioned mechanisms would tend to favor nanospheres as being the most inhibitory due to small size and high surface area to weight ratio.

Bacterial Growth Inhibition

The hypothesis is thus examined with planktonic growth of Methicillin resistant Staphylococcus aureus (MRSA) in the presence of various concentrations ranging from 0.1 μM to 4 μM of ZnO nanoplates, nanospheres, and nanopyramids. Methicillin resistant Staphylococcus aureus (MRSA) subspecies COL was used in this study. All strains are stored in glycerol at −80° C. and plated on tryptic soy agar, cultured overnight at 37° C. and stored at 4° C. Single colony inoculates are grown in TSBG (Tryptic Soy Broth+1% glucose w/v (Sigma)) under aerobic conditions for 16 hours at 30° C. and diluted 1:50 in ZnO NP suspensions to initiate planktonic growth experiments. The optical density at 600 nm is measured every hour for 10 hours and normalized to the initial (T=0) value. For quantitative cultures, 10× serial dilutions of the 10 hour culture are plated and grown for 36 hours at 37° C. to enumerate the number of colony forming units (CFUs) per ml.

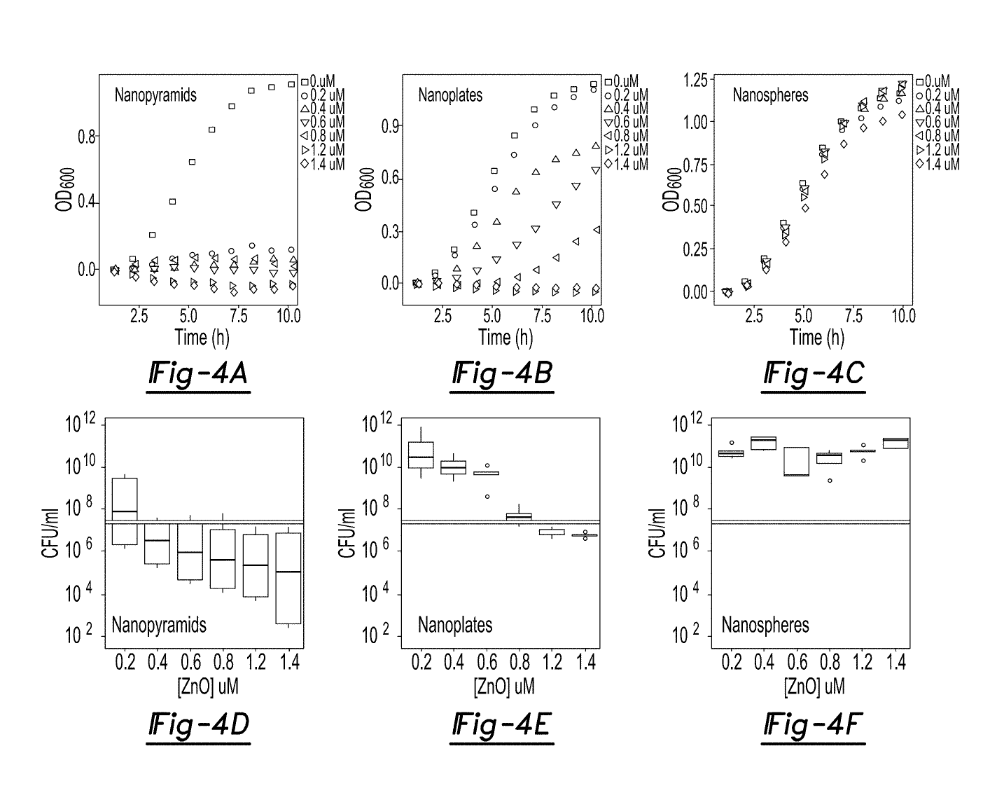

Bacterial growth is inhibited by ZnO NPs in a shape-specific pattern identical to that for GAL inhibition (see FIGS. 4A-4C). The pyramids had near-complete inhibition at all concentrations tested. Nanoplates showed a dose dependent inhibition and spheres showed almost no inhibition.

To confirm the role of NP geometry in antibacterial activity and avoid potential interference from the slight turbidity of NP dispersions, the actual colony forming units (CFUs) are also enumerated for a subset of the planktonic cultures in the presence of each for NP shape (FIGS. 4D-4F). Unlike the other shapes, ZnO nanopyramids had bactericidal function at higher doses. This set of experiments provides additional support to the strong shape-dependence of the antibacterial function of ZnO NPs and the role that enzyme inhibition may play in it.

For any concentration, ZnO nanopyramids prepared in accordance with the present teachings show much higher inhibition ability to GAL, as compared with those of nanoplates and nanospheres. From the investigation of enzyme kinetics such as Michaelis-Menten equation, Lineweaver-Burk, and Eadie-Hofstee analysis, it is found that ZnO nanoplates and nanopyramids follow competitive and noncompetitive (or mixed) enzyme inhibition mechanisms, respectively, while nanospheres appear to have little effect on GAL activity. The shape-dependent inhibition behavior is associated with several factors determining the association of NPs and proteins with geometrical match between the enzyme surface around active center and ZnO nanopyramids being such a factor. Such an inhibition mechanism is not believed to be very enzyme specific, which differentiates biomimetic NP inhibitors from biological inhibitors possessing lock-and-key molecular match with enzyme. While being a potentially limiting factor for some biomedical areas, the mechanisms of inhibition for certain ZnO NPs prepared in accordance with the present disclosure enable their use as broad spectrum inhibitors, for instance, for bacterial enzymes bearing structural similarities to GAL. Considering the fact that the rate of MRSA infections has risen 12-fold for the last decade and is spreading now from hospitals to community outbreaks, a broad spectrum antibacterial resilient to potential mutations of the bacteria altering molecular structure of the typical drug targets is much needed. The fundamental findings and applications presented here represent a shift in the conceptualization of NPs in biological systems from delivery vehicles to nanoscale biomimetic entities with distinct biological function.

The present disclosure thus contemplates nanoparticles comprising a zinc oxide material that form antimicrobial materials. In certain variations, the antimicrobial material comprises nanoparticles provided in a suspension further comprising a liquid (e.g., a carrier). In other variations, the antimicrobial materials may be coatings comprising nanoparticles. In certain variations, the antimicrobial material may be in the form of a coating that is formed via a layer-by-layer process coating. Such antimicrobial materials can minimize or inhibit bacterial growth, including inhibiting growth of gram-positive bacteria (e.g., Staphylococcal growth) and gram-negative bacteria.

Zinc oxide nanoparticles (ZnO-NPs) possess anti-microbial properties, including microbial selectivity, stability, ease of production, and low cost. Zinc oxide, in contrast to silver, is significantly less expensive. This is important because the use of rare materials in disposable medical devices can be cost prohibitive. In addition, the therapeutic window between efficacy and toxicity for silver is quite narrow. This has led to disappointing clinical effectiveness of silver-coated medical devices. ZnO-NPs appear to have improved selectivity for bacteria over mammalian cells. In fact, ZnO is generally recognized as safe by the Federal Drug Administration. In comparison to antimicrobial peptides, which have also been evaluated extensively for this purpose, ZnO-NPs are more stable, easier to prepare, and again significantly less expensive. This makes them a much more attractive alternative for device manufacturers who must consider the costs of regulatory approval and constraints of diminishing health care reimbursements. In this regard, ZnO NPs prepared in accordance with certain aspects of the present disclosure are especially attractive alternatives to silver nanoparticles or antimicrobial peptides for device coatings. Thus, antimicrobial materials incorporating zinc oxide nanoparticles per the present teachings can be used to inhibit microbial growth and to minimize or prevent medical device infection.

Better understanding certain aspects of ZnO NPs efficacy would be beneficial. For example, better understanding the antimicrobial spectrum of ZnO NPs would be beneficial. Given that nanoparticles must come into contact with or touch the bacterial surface to work, it would also be helpful to understand how microbial surface chemistry and nanoparticle shape contribute to ZnO-NP antimicrobial function. Further, substantiating that ZnO-NPs still provide anti-bacterial function when immobilized to a surface is investigated here, especially because surface roughness could increase by the inclusion of ZnO nanoparticles (and potentially increase bacterial adhesiveness) of surfaces formed from standard device fabrication methods. In accordance with certain aspects of the present disclosure, these are addressed to move ZnO-NPs forward as an alternative new anti-infective material (e.g., a coating for implanted medical devices), which is an alternative to silver and other low-molecular weight antimicrobials.

In this example, first the relative efficacy of ZnO NPs against a bacterial suspension (a standard method for establishing antibiotic effectiveness) of Staphylococcus aureus, Staphylococcus epidermidis, Escherichia coli, and Klebsiella pneumonia is explored. These organisms are chosen as they are the two most common Gram-positive and Gram-negative organisms, respectively, recovered from blood cultures in the University of Michigan Hospital Emergency Department annually. Second, how bacterial surface properties (hydrophobicity and acid-base chemistry) relate to ZnO-NP effectiveness is explored. Third, as ZnO particles can be synthesized in various shapes, the effectiveness of certain shapes as antimicrobial agents is also investigated in this example. Whether some other feature of the NP (e.g., shape, surface area) rather than simple mass concentration may be the determining factor in the dose-response relationship is explored. Fourth, when deposited on an in vitro model of a medical device surface, whether ZnO-NPs convey protection to bacterial contamination and biofilm development is also investigated herein.

Therefore, the following examples and discussion explore and evaluate (1) the relative efficacy of ZnO-NPs on planktonic growth of medically relevant pathogens; (2) the role of bacterial surface chemistry, measured by microbial adhesion to solvents, on ZnO-NP effectiveness; (3) NP shape as a factor in the dose-response; and (4) layer-by-layer (LBL) ZnO-NP surface coatings by Calgary biofilm assay. ZnO-NPs inhibit Gram-positive bacterial growth in a shape-dependent manner with the relative inhibition efficacy based on the following order of shapes: pyramids>plates>spheres. Differential susceptibility of various pathogens may be related to their surface hydrophobicity. LBL coatings of ZnO-NP prepared in accordance with certain aspects of the present disclosure reduce Staphylococcal biofilm burden by greater than or equal to about 95%.

### Example 2

Bacterial Strains, Media, and Growth Conditions

The bacterial strains used in this study are Escherichia coli UTI89 and MG1655, Klebsiella pneumoniae LM21, methicillin-resistant Staphylococcus aureus SH1000, and Staphylococcus epidermidis RP62A. Glycerol stocks of all strains maintained at −80° C. are plated on tryptic soy agar, cultured overnight at 37° C. and stored at 4° C. Single colony inoculates are grown in tryptic soy broth+1% glucose w/v (TSBG) under shaking conditions for 16 hours at 30° C. and diluted 1:50 for planktonic growth curves and Calgary biofilm experiments.

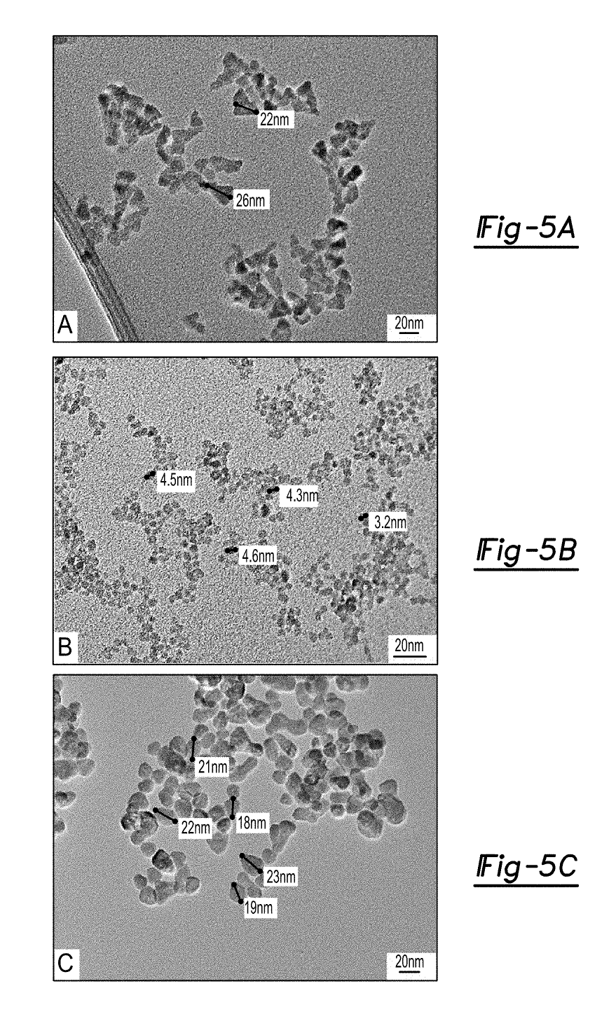

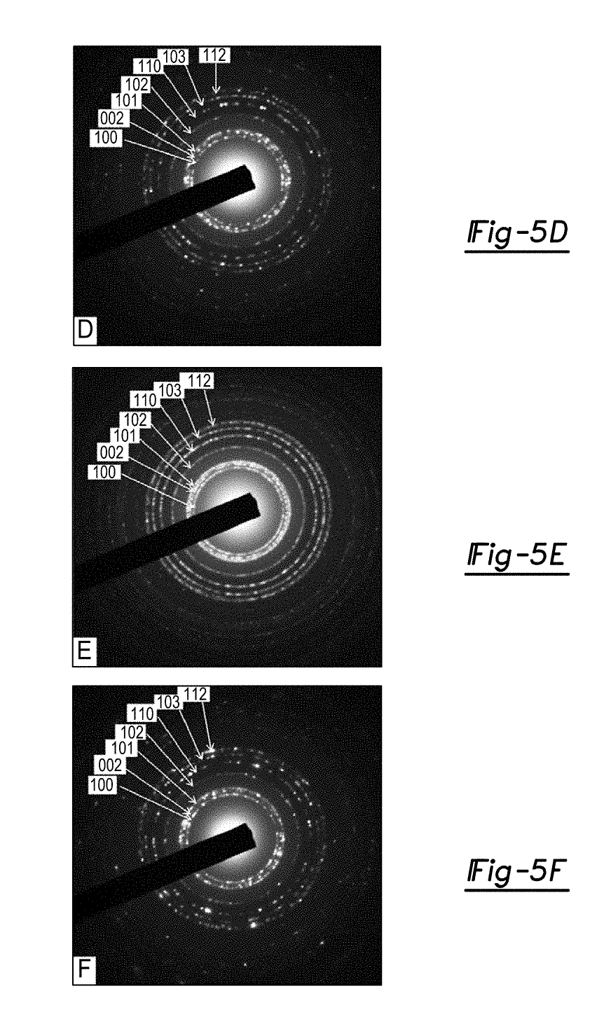

ZnO-NP synthesis: ZnO-NPs are synthesized into three specific shapes, hexagonal pyramids (FIGS. 5A and 5D), plates (FIGS. 5C and 5F), and spheres (FIGS. 5B and 5E). The various shapes are prepared using similar reactions without the use of surfactants or capping agents in order to minimize the effect of different surface chemistry and surface distribution of those molecules on the interaction with the bacterial cell surface. Briefly, plates are synthesized by dissolving 5.5 g Zn(Ac)₂.2H₂O in 100 mL anhydrous methanol and heated to reflux for 1 hour. Then 1 g KOH dissolved in 10 mL deionized water is added to the solution and then refluxed for 14 hours. Sphere synthesis was similar, but the KOH was dissolved in anhydrous methanol instead of deionized water. Pyramids are synthesized by first mixing 0.2 g KOH with the 5.5 g Zn(Ac)₂.2H₂O, before adding anhydrous methanol and refluxing for 48 hours. All NP precipitates are washed 3 times with anhydrous methanol and stored in the freeze-drier.

Characterization of ZnO-NPs:

ZnO-NP preparations are initially characterized by dynamic light scattering (DLS) using a Malvern Instruments Zetasizer Nano ZS to determine size distribution and zeta potential. However, the spherical NPs are quite small (<4 nm average particle size diameter) which limited the accuracy of this method. Repeated DLS measurements of the spheres varied from 40 nm-100 nm. This overestimation compared to transmission electron microscopy (TEM) is likely a function of surrounding water shell and particle aggregation. Therefore, further DLS measurements for the spheres are abandoned. Detailed size measurements and selected area electron diffraction patterns of the ZnO-NPs are made using a JEOL 3011 Transmission Electron Microscope. The samples are prepared by dropping the aqueous solution onto carbon TEM grid and drying at room temperature. In addition, photoluminescence spectra are obtained on a Jobin Yvon Horiba Fluoromax-3 instrument.

### Bacterial ZnO-NP Dose Response Growth Curves

ZnO-NP suspensions are prepared by sonicating ZnO-NPs into TSBG for 30 minutes. Bacterial growth is assessed by optical density at 600 nm (OD₆₀₀) hourly for 10 hours in the presence of ZnO-NPs. To summarize individual growth curves, a growth rate constant is calculated as the slope of the linear portion (i.e., exponential phase of growth) of the log₂(OD₆₀₀) versus time data determined by linear regression.

### Microbial Adhesion to Solvents (MATS) Assay

The MATS assay has been previously described in Bellon-Fontaine, et al., “Microbial Adhesion to Solvents: A Novel Method to Determine the Electron-Donor/Electron-Acceptor or Lewis Acid-Base Properties of Microbial Cells,” Colloids and Surfaces B: Biointerfaces, 7(1-2), pp. 47-53 (July 1996). Bacteria are grown overnight in tryptic soy broth (TSBG) media, pelleted, and resuspended in phosphate buffered saline (PBS) to OD₆₀₀ of 0.6 for stationary phase. For mid log phase the overnight culture is diluted 1:50 and grown for 4 hours prior to pelleting and resuspension at OD₆₀₀ of 0.6. Bacterial cell suspensions (1.2 ml) are vortex mixed for 90 seconds with various solvents (0.2 ml). The mixture is allowed to stand for 15 minutes to ensure complete separation of the two phases before a sample is carefully removed from the aqueous phase and the OD₆₀₀ measured. The percentage of bound cells is subsequently calculated by:

$$Partition Fraction = ( 1 - \frac{A}{{A}_{0}} ) \times 100$$

where A₀ is the OD₆₀₀ of the bacterial suspension before mixing and A is the OD₆₀₀ after mixing.

To determine hydrophobicity, the hydrophobic solvent hexadecane is used. The fraction of cells that partition to the hexadecane-aqueous interface is a measure of cell surface hydrophobicity. For the Lewis acid-base properties, a comparison between microbial cell migration to the solvent-aqueous interface for a monopolar (acidic or basic) solvent and an apolar solvent is made. Increased affinity for chloroform-aqueous interface over hexadecane aqueous interface is a measure of cell surface electron donating properties (e.g., Lewis base). Increased affinity for diethyl ether-aqueous interface over a hexane-aqueous interface is a measure of cell surface electron accepting properties (e.g., Lewis base).

### Layer-by-Layer (LBL) ZnO-NP Surface Coating

96-well plate lids fit with polystyrene pegs (e.g., cylindrical posts as part of a Calgary Biofilm Device) are coated with ZnO-NPs. Pegs are prepared using the UVO Cleaner (Jelight). ZnO-NP suspensions are prepared by dissolving the appropriate NP in deionized water to a concentration of 0.1% w/v. Polystyrene sulfonate (PSS) is dissolved in deionized water to a concentration of 5% w/v. Prepared pegs are placed into NP suspensions for 30 minutes, rinsed with deionized water and quickly blown dry with nitrogen. Pegs are then placed in PSS solution for 5 minutes, rinsed and dried again. They are returned to NP suspension, and the process is repeated 10 times with the final coating being NPs.

### Characterization of LBL ZnO-NP Coatings

Adsorption of ZnO in each layer is confirmed by UV-vis spectroscopy using an 8453 UV-vis ChemStation Photospectrophotometer (Agilent Technologies). Completed LBL coatings are characterized by scanning electron microscopy (SEM) and atomic force microscopy (AFM). For SEM, samples are fixed in glutaraldehyde, serially dehydrated in ethanol, air dried at room temp, sputter-coated with gold and visualized using AMRAY 1910 Field Emission Scanning Electron Microscope. For AFM, samples are imaged in tamping mode using the Asylum Research MFP-3D atomic force microscope. The NCH Pointprobe cantilevers by Nano World with a nominal spring constant and resonance frequency of 42 N/m and 320 kHz, respectively are used. Roughness analysis of the AFM images is performed using the Asylum Research software. Goniometry measurements are taken using high resolution photographs of the contact angle between water and the LBL coated surfaces.

ZnO-NP Leaching from LBL Surfaces:

To confirm stability of the LBL coatings, polystyrene pegs (e.g., cylindrical posts) with ZnO-NP LBL coatings are incubated in either sterile water or PBS for a period of 7 days. ZnO leaching is quantified by absorbance at 350 nm (A₃₅₀) of the surrounding medium and compared to the ZnO-NP suspension used for the coating process (positive control) and uncoated polystyrene pegs (negative control).

### Conversion of Mass Concentration to Surface Area and Particle Number Concentration

For each NP shape an idealized geometry was assumed and the corresponding volumes and surface areas were calculated. The particle number concentration was calculated as:

$$[ Partice \# ] = \frac{[ Mass ]}{{V}_{ρ}}$$

where V is the particle volume and ρ is the density of ZnO (5.6 g/cm³). The surface area concentration was calculated as:

[Surface Area]=[Particle #]S_{A}

where SA is the particle surface area.

### Bacterial Surface Colonization Assay

Bacterial surface colonization is evaluated using the Calgary Biofilm Device. LBL ZnO-NP coated pegs are submerged in inoculated media for 16 hours at 37° C. The pegs are removed, washed twice, and then sonicated for 10 minutes to liberate adherent bacteria. Quantitative culture is then performed to determine the colony forming units (CFUs) present on each peg. The limit of detection for this assay is 100 CFUs per square centimeter of peg. Pegs are also prepared for SEM.

### Statistics

All data is presented as mean plus or minus standard error of the mean unless otherwise noted. For the MATS assay, experiments are performed in triplicate. Two-way ANOVA is performed with bacterial strain and growth phase as factors. For the Calgary biofilm experiments, one-way repeated measures ANOVA is performed for each bacterial strain to evaluate the effect of particle shape on bacteria recovered from the biofilms. In all cases, post-hoc pairwise testing is performed using the Tukey procedure with significance set at p<0.05.

For the planktonic growth curve experiments, linear mixed effects regression is performed with log transformed optical density as the dependent variable, time as a fixed effect, and date of experiment as a random effect to calculate the growth rate constant. For comparison of dose response curves, linear mixed effects regression I is again performed with growth rate constant as the dependent variable, time and shape as fixed effects, and date of experiment as a random effect. Reported p-values represent the significance of shape as a predictor of the dose response.

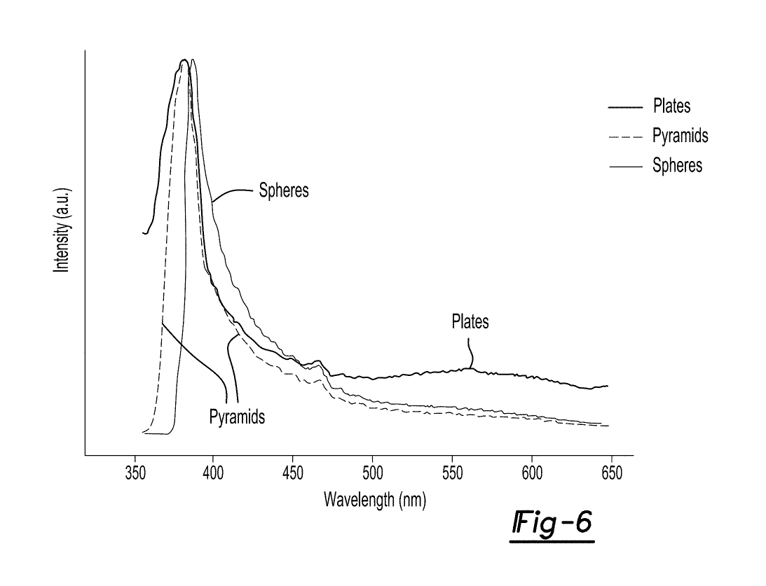

In this example like in Example 1, three ZnO-NP geometries are studies: plates, spheres, and pyramids. The edges of the hexagonal base of pyramids are an average of approximately 20 nm, while side edges are approximately 25 nm (FIG. 5A). The average diameter of spheres is approximately 4.4 nm (FIG. 5B). The average diameter and average thickness of plates are approximately 20 nm and approximately 3.5 nm, respectively (FIG. 5C). Despite the obvious differences in the shape of the NPs, the crystals structures are nearly identical and all diffraction rings could be matched to the hexagonal phase of bulk ZnO (JCPDS 36-1451)(FIGS. 5D-5F). In addition, photoluminescence spectra confirmed minimal differences in surface chemistry among the three NP shapes (FIG. 6).

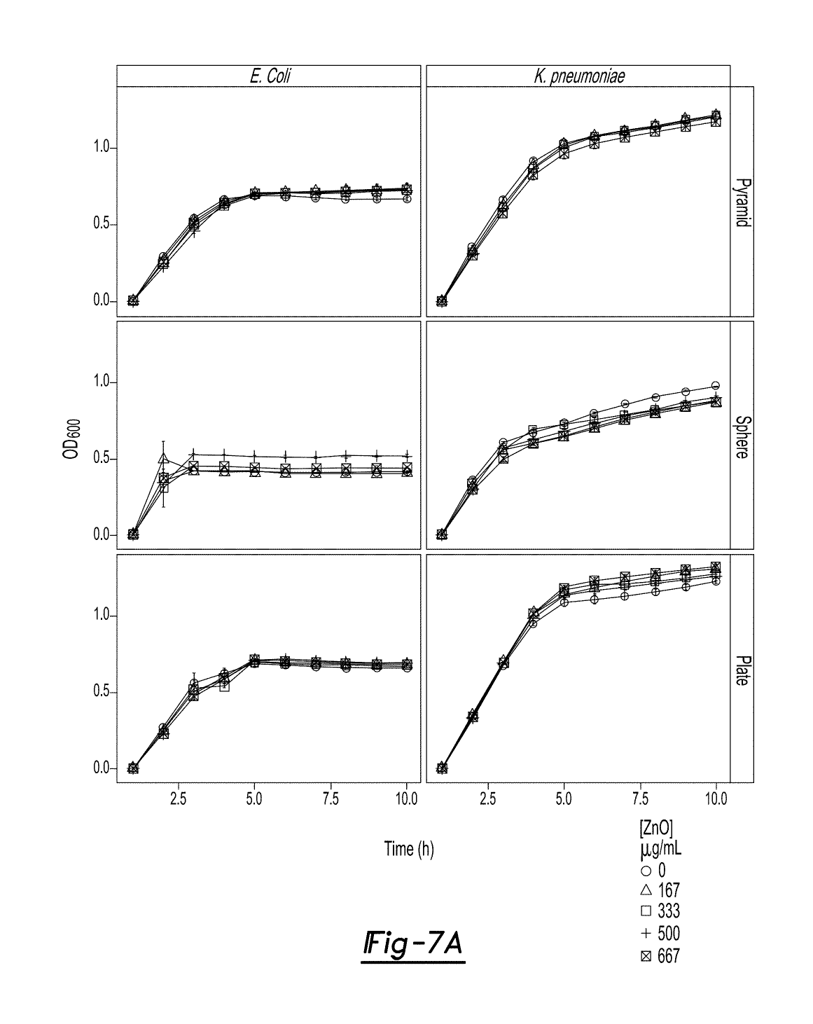

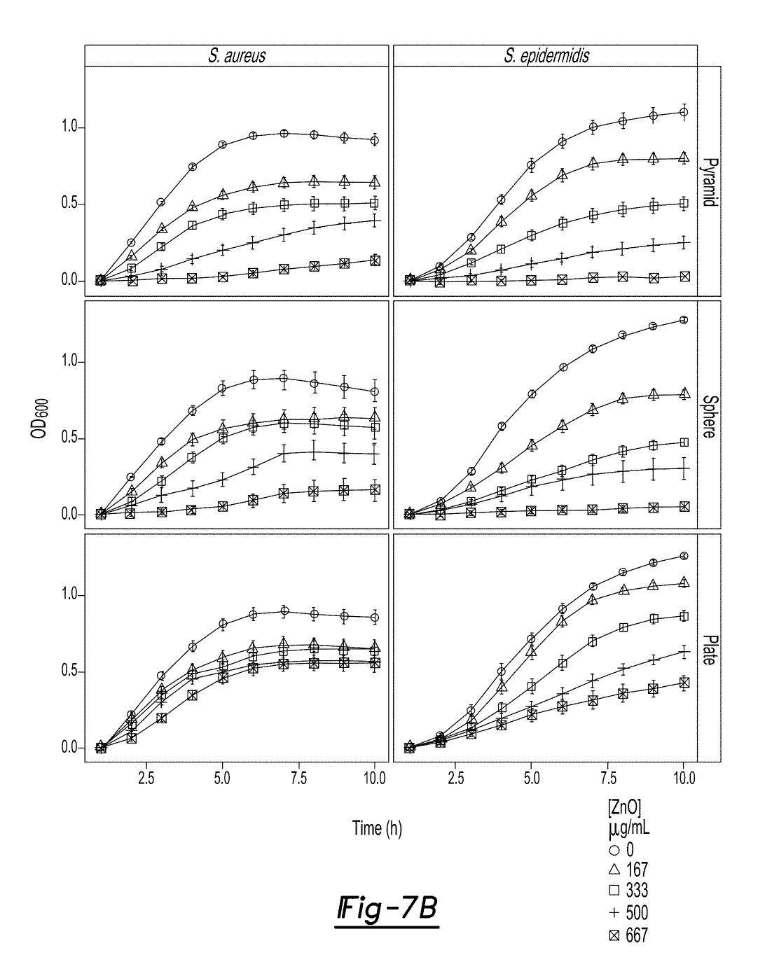

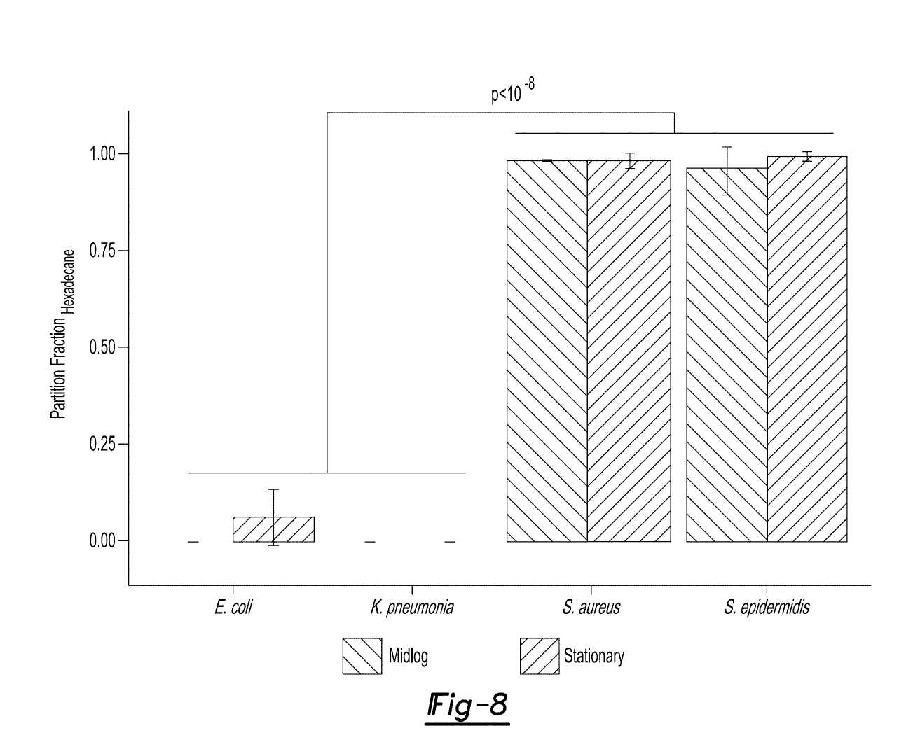

Planktonic growth curves are generated for each bacterial strain in the presence of escalating mass concentrations of each ZnO-NP shape (FIGS. 7A-7B). The Gram-positive organisms (i.e., S. aureus and S. epidermidis in FIG. 7A) show a dose-dependent reduction in growth for all three NP shapes. The Gram-negative organisms (i.e., E. coli and K. pneumonia in FIG. 7B) are not affected by the presence of ZnO-NPs up to 667 μg/ml.

To examine the role of bacterial surface chemistry on ZnO-NP antibacterial function, the well-established microbial adhesion to solvents assay (MATS) is used to determine the surface hydrophobicity (FIG. 8) and Lewis acid-base properties of the four test organisms. The two Gram-positive species tested are found to be highly hydrophobic, with near complete migration (99%±1% for S. epidermidis and 98%±2% for S. aureus) to the aqueous-hexadecane interface. The opposite is observed with E. coli and K. pneumoniae, for which no more than 10% (6.6%±7% and 0% respectively) of suspended organisms are found at the solvent interface after extensive mixing. There are no significant differences in hydrophobicity measured during mid-log versus stationary phase of growth.

The Gram-positive organisms have extreme hydrophobicity and therefore minimal Lewis acid-base interactions. E. coli and K. pneumonia have similar partitioning to the chloroform-aqueous interface during mid-log growth indicating a modest electron donating capacity on their surface (29%±2% and 24%±7% respectively). During stationary phase, E. coli have increased migration to the chloroform-aqueous interface (71%±7%) while K. pneumonia have a slight but insignificant decrease (20%±2%) when compared to mid-log phase. E. coli and K. pneumonia also have similar migration to the diethyl ether-aqueous interface at mid log (33%±9% and 30%±2% respectively) and stationary phase (28%±1% and 28%±7% respectively) indicating modest electron accepting capacity.

To summarize the growth curve data in FIGS. 7A-7B make direct comparisons of the three NP shapes, a growth rate constant is calculated for each dose of each NP shape. To determine if some other feature of the NP (e.g., shape, surface area) may be the determining factor in the dose-response, the mass concentrations used for the planktonic growth curves are converted to surface area and molar concentrations based on the TEM measurements (FIGS. 5A-5C) and known density of ZnO. While the same mass of pyramid, plate, and sphere NPs is used in each experiment, those masses converted to large differences in available surface area and total particle number.

TABLE 1
ZnO-NP concentrations for different dosing units

| mass | [surface area] m²/L | | | [particle #] nM | | |
|---|---|---|---|---|---|---|
| μg/mL | sp* | pl^{†} | py^{‡} | sp | pl | py |
| 167 | 44.7 | 1.36 | 0.69 | 1478 | 45 | 23 |
| 333 | 89.2 | 2.72 | 1.38 | 2948 | 90 | 46 |
| 500 | 134 | 4.08 | 2.07 | 4426 | 135 | 69 |
| 667 | 179 | 5.45 | 2.77 | 5904 | 180 | 91 |

*spheres,
^{†}plates,
^{‡}pyramids

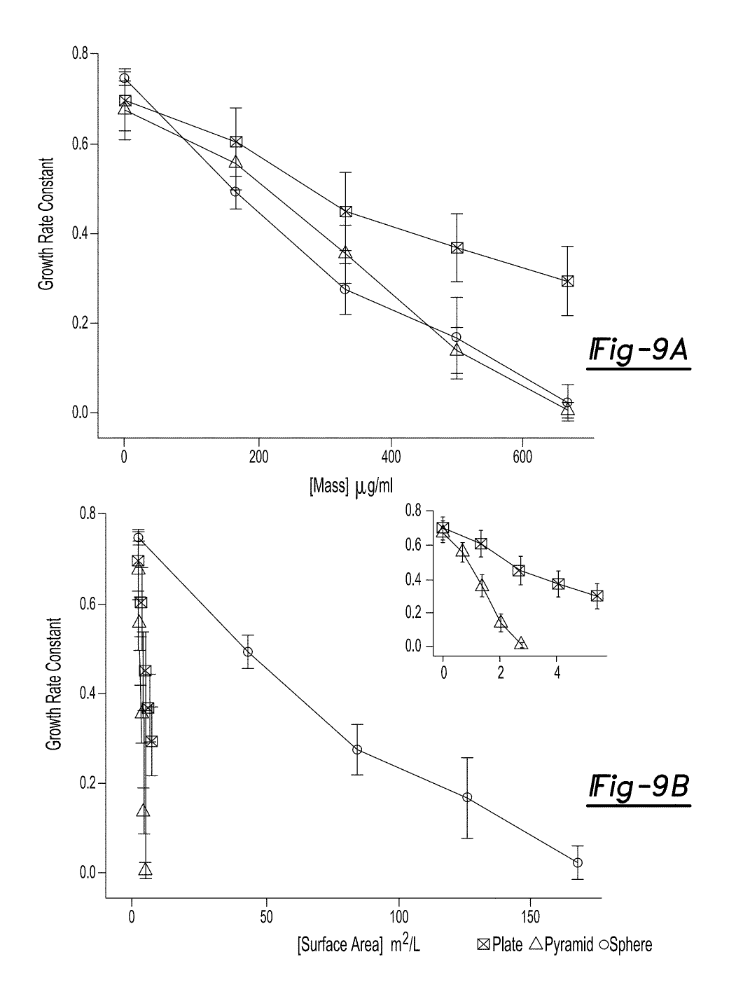

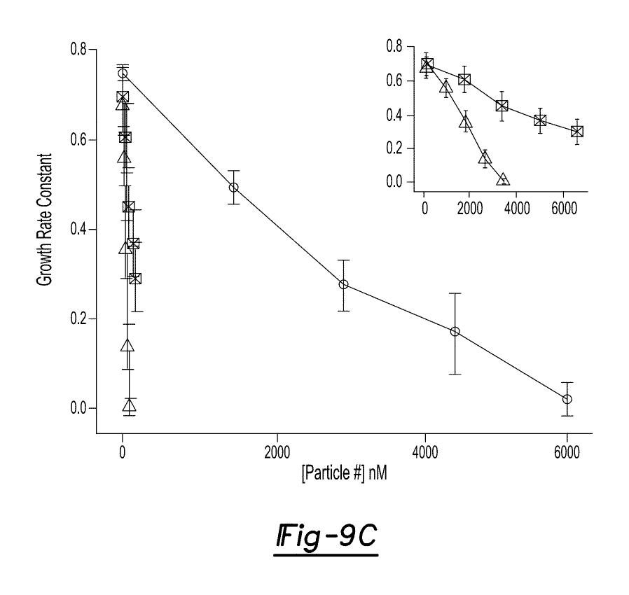

As a quantitative measure of the antibacterial effect of a certain dose and specific shape, the growth rate constant is plotted against the mass, surface area, and particle concentrations for each of the three NP shapes (FIGS. 9A-9C). The effectiveness of a given dose is determined by the reduction in growth rate constant. For mass concentration, the dose response for spheres and pyramids are essentially equal (p=0.96), while plates had a somewhat attenuated effectiveness (p<0.05, FIG. 9A). However, for surface area and particle number concentration, pyramids had the greatest dose response followed by plates and then spheres (p<10⁻³, FIGS. 9B and 9C).

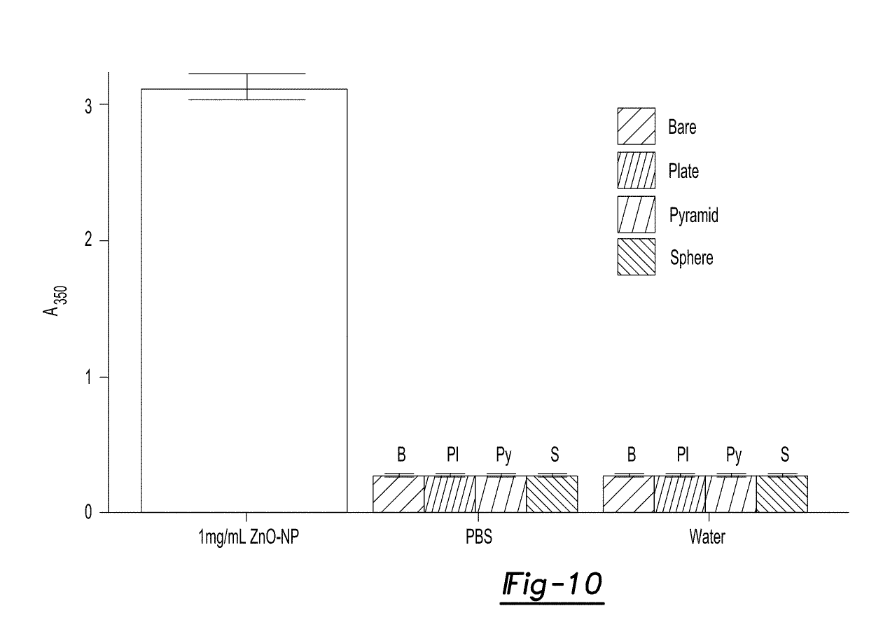

The application of ZnO-NPs to surfaces using the LBL technique is confirmed by UV-vis spectroscopy. The increase in absorption at 350 nm per layer varied between 0.04-0.05 for plates and spheres and 0.02-0.03 for spheres. This difference is thought to be related the fact that the smaller spheres create thinner ZnO layers and therefore less absorption. Final LBL surface coatings of ZnO-NPs are characterized by SEM, AFM, and goniometry. All ZnO-NP shapes increased roughness over the starting substrate. Contact angles varied from 18° for plates to 56° for pyramids. These coatings remained stable with no measurable leaching of ZnO into PBS or water over 7 days (FIG. 10).

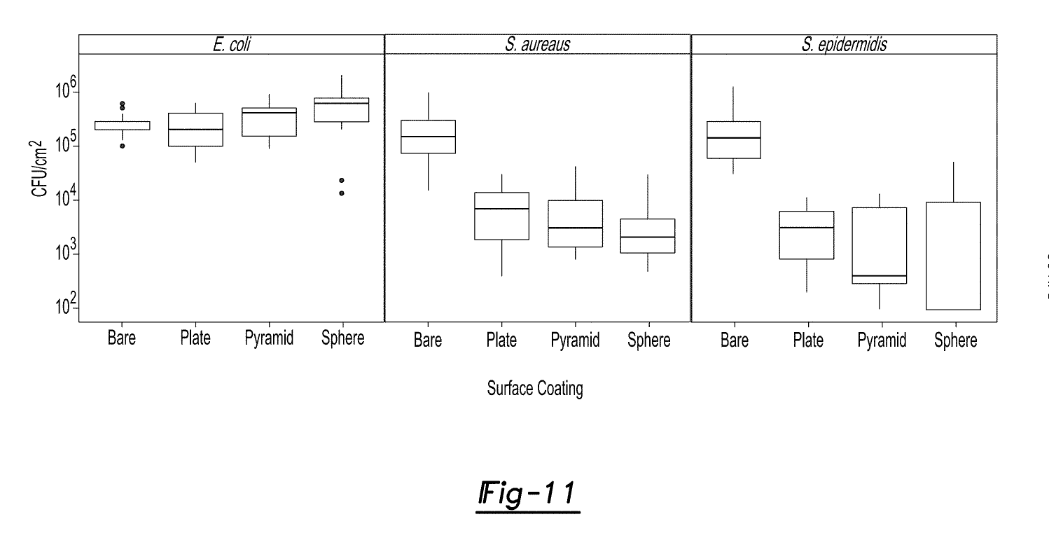

Despite the increased surface roughness, a dramatic reduction in bacterial burden on polystyrene coated with ZnO-NPs is demonstrated. The coated surfaces had ≥95% (p<10⁻³) reduction in the number S. aureus and S. epidermidis, but not E. coli cells recovered when compared to bare surfaces (FIG. 11). There are no significant NP shape effects on biofilm inhibition of S. aureus. On the other hand, spheres had a significantly greater inhibitory effect over plates for S. epidermidis (99.5% vs 98.5%, p of approximately 0.005).

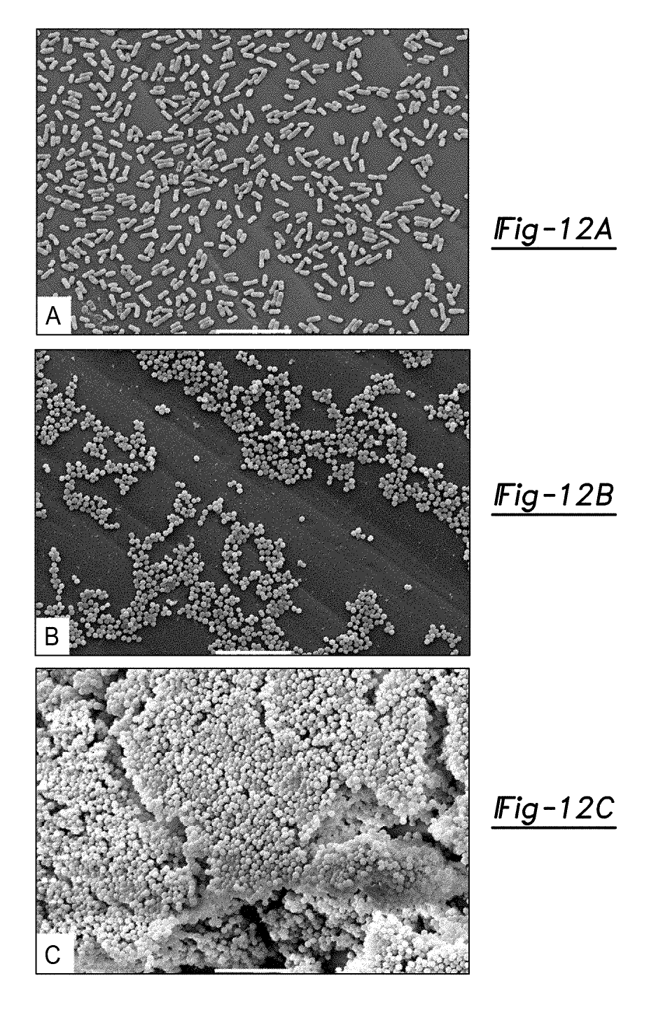

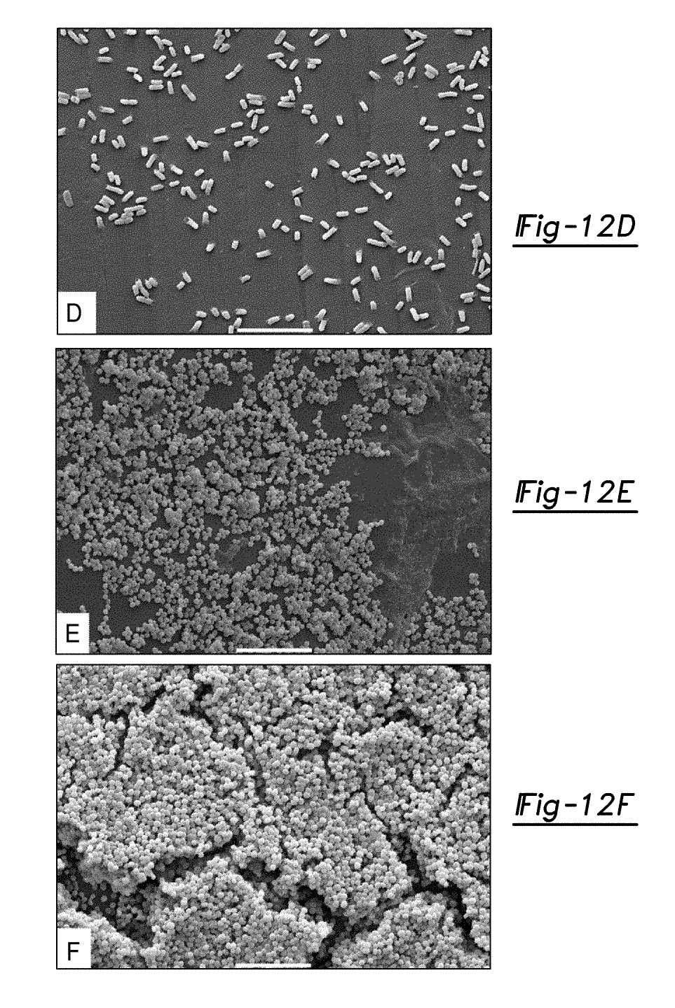

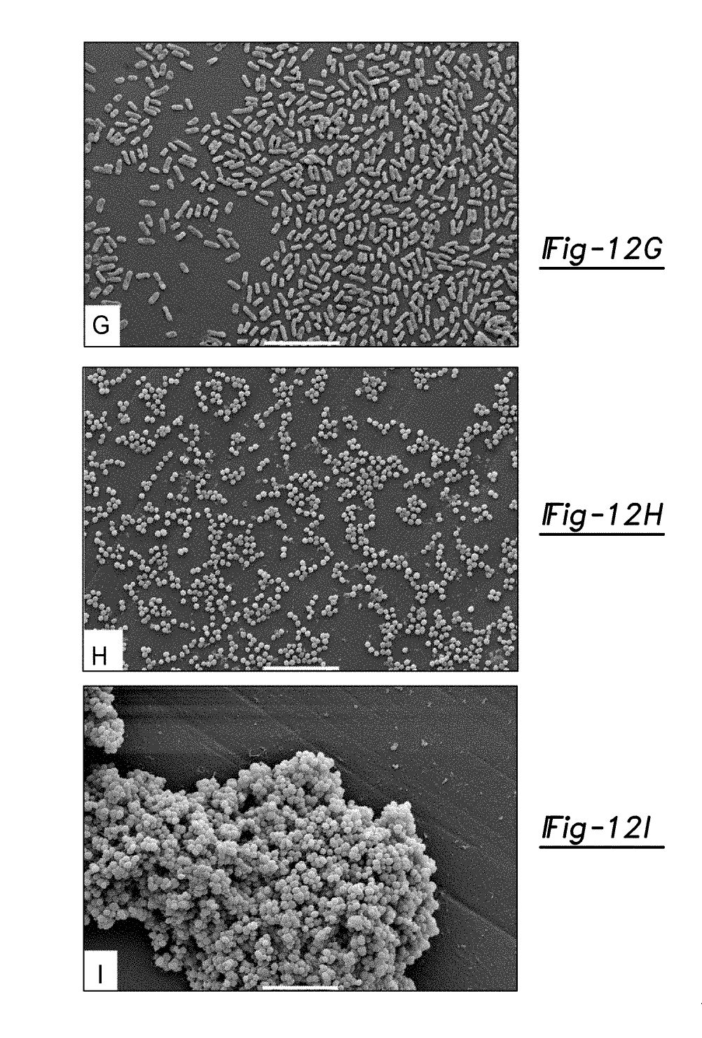

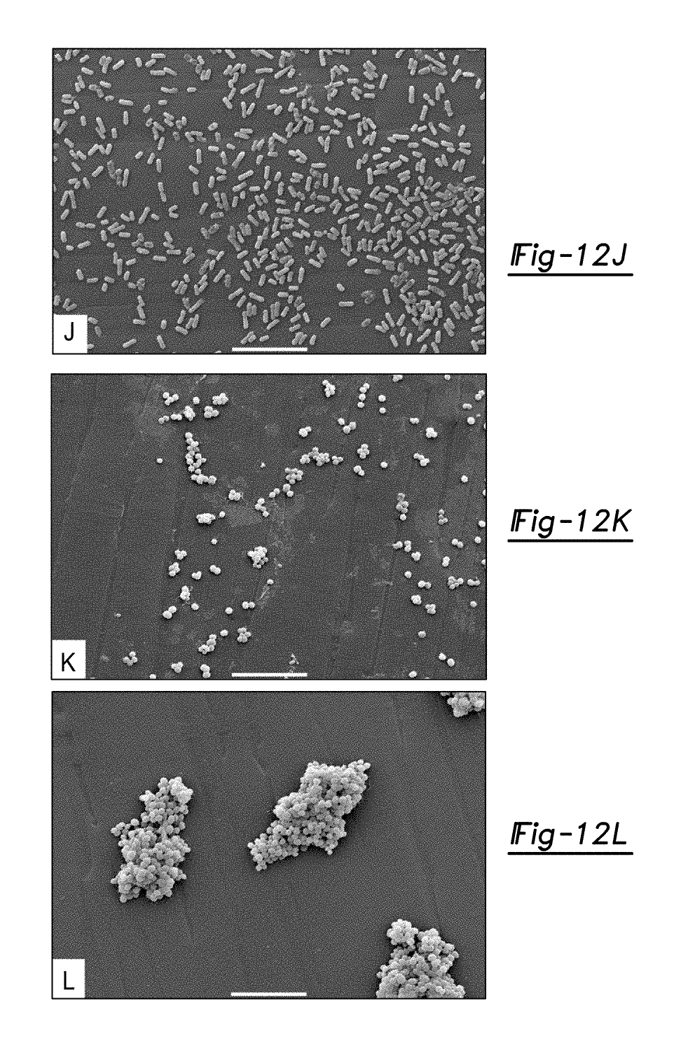

To better understand how the bacteria are interacting with the surfaces, SEM is performed on surfaces after biofilm culture (FIGS. 12A-12L). These images demonstrate bacterial adhesion and biofilm development on bare surfaces (FIGS. 12A-12C) particularly for S. epidermidis (FIG. 12C). However, despite an almost 3 log reduction in the number of viable cells recovered from the ZnO-NP coated surfaces (FIG. 11) there continues to be intact cells visualized on SEM (FIGS. 12D-12L). In particular, there is a 99.5% reduction in S. epidermidis recovered from the ZnO sphere coated surface, but persistence of biofilm appearance on SEM (FIG. 12F).

In accordance with the present teachings, ZnO-NPs are a new antimicrobial technology with many features that make them an attractive alternative to silver or antimicrobial peptides for preventing medical device infection. ZnO-NPs can be synthesized into various distinct shapes without the use of traditional surfactants or capping agents. This feature of the synthesis process is significant in light of the potential for these additional molecules to confound the results of experiments. As such, ZnO-NPs with high crystallinity are synthesized with nearly identical surface chemistry, differing only in shape and size.

One hypothesis is that four test organisms (including Gram-positive, Gram-negative, and spore forming organisms) would be susceptible to the ZnO-NPs prepared in accordance with the present teachings. Suspensions of ZnO-NP prepared in accordance with certain aspects of the present disclosure selectively inhibit the growth of Gram-positive organisms including methicillin resistant S. aureus (MRSA). While other conventional ZnO-NPs have also shown a dose-dependent selectivity of ZnO-NPs for Gram-positive organisms, there are multiple studies demonstrating growth inhibition of Gram-negative organisms including E. coli. In the current example, dose-dependent selectivity with Gram-negative organisms was not observed with the ZnO-NPs. Without limiting the present technology to any particular theories, the discrepancy can be attributed to three possible phenomena. The first is the use of surfactants and capping agents for NP synthesis in conventional methods of forming ZnO particles, which likely change the surface energies of both bacteria and particles and therefore modulate the free energy of interaction. Indeed, the ZnO-NP synthesis media used by Brayner et al., “Toxicological Impact Studies Based on Escherichia coli Bacteria in Ultrafine ZnO Nanoparticles Colloidal Medium,” Nano Letters, 6(4), pp. 866-70 (April 2006) led to membrane disruption in the absence of nanoparticles.

Second, in many cases, previously tested strains of E. coli are laboratory strains or expression vectors that lack the clinically ubiquitous capsule or surface proteins which may provide protection against the ZnO-NPs. Finally, the molar dose of ZnO-NPs used in previous studies is much larger (1-6 mM) than that used here (23 nM-6 μM). Gram-negative organisms may require a higher particle number for bacterial inhibition. Reddy et al, “Selective toxicity of zinc oxide nanoparticles to prokaryotic and eukaryotic systems,” Applied Physics Letters, 90(21) (2007) showed that E. coli required >3.4 mM ZnO-NPs for complete inhibition whereas S. aureus only required 1 mM. However, these higher concentrations may be toxic to mammalian cells. For instance, human T-cells begin to show toxicity to ZnO-NPs at concentrations around 5 mM. The effective concentrations used here are at least 1000-fold lower.

The current conventional understanding and consensus in the art is that ZnO-NPs work through contact with the bacterial surface, which leads to either cell membrane disruption or generation of reactive oxygen species. Assuming this mechanism to be true, the surface chemistry of a bacterium will likely influence the potential for interaction between the NP and the bacterium. To that end, how bacterial surface chemistry may contribute to ZnO-NP susceptibility has been investigated. The striking contrast in surface hydrophobicity between Gram-positive and Gram-negative organisms parallels their susceptibility to ZnO-NPs. Furthermore, it provides evidence for a mechanism by which surfactants or capping molecules alter surface interactions and thereby antibacterial function of conventionally formed ZnO-NPs. Of note, the presence of both electron donating and accepting properties in the Gram-negative organisms have been observed. This is likely a result of the heterogeneous composition of the cell surface and the presence of zwitterions. Therefore, the acid-base surface chemistry of a cell is a function of the pH of the surrounding media. All experiments in this case are performed with normal PBS at pH 7.4.

Decreasing particle size (and increasing affective surface area) leads to increase antimicrobial efficacy, while differences in particle shape may alter function to a much lesser degree. However, it can be difficult to sort out the relative contributions of size and shape. This is further confounded by the use of different capping agents to modify particle shape. Stankovic et al., “Influence of size scale and morphology on antibacterial properties of ZnO powders hydrothermally synthesized using different surface stabilizing agents,” Colloids and Surfaces B, Biointerfaces, 102, pp. 21-28 (February 2013) showed that the antibacterial activity of ZnO-NPs depends on their synthesis method and the resulting morphology and surface area. However, a variety of stabilizing agents were used that may have contributed to the observed differences. The present technology provides an ability to synthesize three different NP shapes with similar crystalline structure and surface chemistry, which better allows investigation of the role of shape in the antibacterial properties of ZnO-NPs.

In general, when shape and size have been considered, the smallest NP, regardless of shape, appears to be the most effective. Therefore, it is hypothesized that the ZnO-NP spheres which are small and have high surface area to weight ratio would be the most effective. However, it has been discovered that ZnO pyramids at the same mass concentration as other shaped particles had greater or equal effectiveness with less surface area and less particle number. This data suggest that shape may play a more important role than previously considered. In light of the previous discussion regarding the modulating effects of stabilizing molecules, previous work comparing different shapes may have been confounded by the synthesis technique. Because the NPs synthesized here are devoid of surfactant or stabilizing molecules (with the exception of KOH which is used in all three preparations) a more direct comparison of shape is possible. Indeed, the most effective NP shape on a molar concentration basis is not the smallest (spheres), rather the largest (pyramids). This brings to light the difficulty in teasing out the independent contribution of size and shape on ZnO-NP antibacterial efficacy and careful consideration of the appropriate units of dose in studies of nanoparticles as medical therapeutics.

Given the capacity of ZnO-NPs to inhibit planktonic bacterial growth, these particles can be used for surface-based biofilm inhibition. A layer-by-layer (LBL) technique can immobilize the ZnO-NPs to a substrate, such as a polymer substrate. This is the first use of LBL ZnO-NPs as an antibacterial coating. This process is simple, inexpensive, and can be applied to many different polymer surfaces used for medical devices.

Layer-by-layer assembly (LBL) provides a reliable method for fabricating coatings with favorable physical characteristics. The LBL technique is well known and relies on alternating adsorption of charged species or polyelectrolytes onto a substrate. Layers are built up by sequential dipping of a substrate into oppositely charged solutions having oppositely charged moieties that are attracted to the surface. Monolayers of individual components attracted to each other by electrostatic and van-der-Waals interactions are thus sequentially adsorbed on the target surface. LBL films can be constructed on a variety of solid substrates, thus imparting much flexibility for size, geometry and shape and further patterned or etched (with chemicals, plasma, electron beam, or high intensity lasers, for example). In preferred aspects, the substrate is polymeric. Additionally, LBL multilayers have both ionic and electronic conductivity that provides favorable charge transfer characteristics.

In an exemplary LBL method, a substrate has a first charge. A first charged material or moiety has a first polarity that is opposite to the charge of the substrate. By way of non-limiting example, the substrate may have a negative charge, while the first charged material has a positive charge. The first charged material is thus applied to substrate in a first step (Step 1), for example, by applying the first charged material onto the regions of the substrate. The driving force is electrostatic attraction. Additional steps may occur between application steps, such as washing of the surface before application of the next material. After application of the first charged material to the substrate, the surface of the substrate can be exposed to a first wash material in Step 2, which is an optional step. Then, a second charged material or moiety having a second polarity opposite from the first polarity is applied over the first charged material in Step 3. Then, the surface having both the first charged material and the second charged material disposed thereon can be exposed to a second wash material in Step 4, which like Step 2 is likewise optional.

Steps 1-4 serve as a single deposition cycle that may be repeated sequentially to build distinct alternating layers of the first charged material and second charged material. A composite material layer comprises the first charged material and the second charged material. Depending on the charge of the substrate, the first charged material may be either a polycation or a polyanion (so that it is attracted to and deposited onto the surface of the substrate). Thus, the second charged material is the other of the polycation or the polyanion, having an opposite charge to the first charged material. Accordingly, a composite coating or material is formed by LBL is often referred to as: (polyanion/polycation)ₙ, where n represents the number of deposition cycles or layers present. LBL thus provides a simple tool for making thin film coating structures having homogeneously dispersed, well organized layered structures with high levels of both polyanion and polycation.

In certain aspects of the present disclosure, a first charged material or moiety is the ZnO-NPs, which have a positive charge and may be a polycation. Of course, as appreciated by those of skill in the art, whether the first charged material is anionic or cationic depends on the material used to form the coating and the substrate charge. Suspensions are prepared by dissolving the appropriate NP in deionized water to a concentration of 0.1% w/v. Polystyrene sulfonate (PSS) is dissolved in deionized water to a concentration of 5% w/v. The second charged material or moiety may be polyanion, poly(sodium 4-styrenesulfonate) (PSS), having a negative charge. The PSS has a strong negative charge that is complementary to the positive charge of ZnO-NPs, permitting layer-by-layer (LBL) deposition to make a multi-layer coating. Furthermore, various negative charged materials can be applied via LBL with the complementary ionic pairing partner of ZnO-NPs to form coatings having the desired properties on the surface of the substrate. It should be noted that desirably the final external layer of the coating comprises ZnO-NPs to facilitate maximal contact with the surrounding environment, including microbes present therein.

In certain other aspects, the present disclosure also contemplates medical devices that comprise the zinc oxide nanoparticles as an antimicrobial material. In certain variations, the coating comprising the ZnO nanoparticles are biocompatible and capable of introduction and/or implantation within an organism, such as an animal. A medical device includes any device that may be implanted temporarily or permanently in a human or other animal. The ZnO-NP containing antimicrobial materials of the present disclosure are particularly suitable for indwelling medical devices. Examples of medical devices include, but are not limited to, catheters, stents, expandable stents, such as balloon-expandable stents, coronary stents, peripheral stents, stent-grafts, other devices for various bodily lumen or orifices, grafts, vascular grafts, arteriovenous grafts, by-pass grafts, pacemakers and defibrillators, leads and electrodes, patent foramen ovale closure devices, artificial heart valves, anastomotic clips, arterial closure devices, cerebrospinal fluid shunts, prostheses, and the like. Thus, the medical device may be intended for any vessel in an animal, including cardiac, renal, neurological, carotid, venal, coronary, aortic, iliac, femoral, popliteal vasculature, and urethral passages, by way of non-limiting example.

NP coatings are likely to increase surface roughness and therefore bacterial adhesion. Indeed, all the ZnO-NP coated surfaces formed are significantly rougher than a comparative bare substrate. However, dramatic reductions (e.g., ≥95%) in the numbers of viable bacteria recovered from ZnO-NP coatings were observed. This is comparable to the antibacterial performance of chlorhexidine-silver sulfadiazine coated catheters currently used clinically. SEM is used to better visualize the interactions of cells with the surfaces. However, it should be noted that SEM is limited in that it cannot differentiate living from dying cells. Given this limitation, cells of unclear viability are shown adhering to the surfaces coated with ZnO-NPs. However, based on the quantitative culture, the cells dispersed from the ZnO-NP coated surfaces are no longer viable (i.e., able to form a colony). That is, the ZnO coated surfaces may promote adhesion, but lead to contact killing. To be sure, SEM images before and after dispersion for quantitative culture demonstrated that the majority of cells are indeed removed from all surfaces and that the quantitative culture results are not biased by the ability to disperse the cells from the surface. Of note, it is possible for the cells to have a viable, but uncultureable phenotype, which could not be differentiated by this analysis.

In conclusion, ZnO-NPs prepared in accordance with certain aspects of the present technology can reduce planktonic growth of Gram-positive in a dose-dependent manner, which may be related in part to bacterial surface hydrophobicity. Shape appears to modulate the dose response for ZnO-NPs, when either particle number or surface area is used as dosing units. LBL coating of polystyrene with ZnO-NP reduces staphylococcal biofilm burden despite increased in surface roughness and likely bacterial adhesion. This work furthers ZnO-NPs as alternative medical device coating materials.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An enzyme inhibitory nanoparticle comprising zinc oxide, wherein the nanoparticle exhibits substantially reversible enzyme inhibition in the presence of an enzyme.

2. The enzyme inhibitory nanoparticle of claim 1, wherein the substantially reversible enzyme inhibition in the presence of the enzyme reduces enzyme activity by greater than or equal to about 75%.

3. The enzyme inhibitory nanoparticle of claim 1, wherein the reversible inhibition in the presence of the enzyme reduces enzyme activity by greater than or equal to about 95%.

4. The enzyme inhibitory nanoparticle of claim 1, wherein the nanoparticle has a non-spherical shape selected from the group consisting of: polyhedrons, pyramids, cones, discs, plates, rods, cylinders, stars, rectangles, and combinations thereof.

5. The enzyme inhibitory nanoparticle of claim 4, wherein the non-spherical shape of the nanoparticle is a pyramid.

6. The enzyme inhibitory nanoparticle of claim 1, wherein the nanoparticle has a maximum dimension of less than or equal to about 20 nm.

7. The enzyme inhibitory nanoparticle of claim 1, wherein the nanoparticle has a maximum dimension of greater than or equal to about 1 nm to less than or equal to about 20 nm.

8. The enzyme inhibitory nanoparticle of claim 1, wherein the nanoparticle has a surface comprising zinc oxide, but the surface is substantially free of capping agents, surfactants, and stabilizing agents other than the potassium hydroxide (KOH).

9. The enzyme inhibitory nanoparticle of claim 1, wherein the nanoparticle exhibits antimicrobial activity in the presence of bacteria.

10. An antimicrobial material comprising:
a layer-by-layer coating comprising a plurality of nanoparticles comprising zinc oxide, wherein each nanoparticle exhibits antimicrobial activity in the presence of bacteria.

11. The antimicrobial material of claim 10, wherein the plurality of nanoparticles comprising zinc oxide is a first layer and the layer-by-layer coating further comprises a second distinct layer comprising a polyanion, wherein the first layer forms an external exposed surface of the layer-by-layer coating.

12. The antimicrobial material of claim 10, wherein the antimicrobial material exhibits antimicrobial activity in the presence of bacteria to reduce a biofilm burden by greater than or equal to about 95% as compared to a surface without the layer-by-layer coating.

13. The antimicrobial material of claim 10, wherein each nanoparticle of the plurality has a non-spherical shape selected from the group consisting of: polyhedrons, pyramids, cones, discs, plates, rods, cylinders, stars, rectangles, and combinations thereof.

14. The antimicrobial material of claim 10, wherein the plurality of nanoparticles has a pyramid shape.

15. The antimicrobial material of claim 10, wherein the plurality of nanoparticles has a maximum dimension of less than or equal to about 20 nm.

16. The antimicrobial material of claim 10, wherein the each nanoparticle of the plurality has a surface comprising zinc oxide, but the surface is substantially free of capping agents, surfactants, and stabilizing agents other than KOH.

17. An in-dwelling medical device comprising the antimicrobial material of claim 10.

18. A method of preparing an enzyme inhibitory or antimicrobial nanoparticles comprising zinc oxide, the method comprising:
reacting a precursor comprising zinc with potassium hydroxide (KOH) in the presence of an alcohol to form a zinc oxide nanoparticle, wherein the nanoparticle has a surface comprising zinc oxide that is substantially free of capping agents, surfactants, and stabilizing agents other than the KOH.

19. The method of claim 18, wherein the zinc oxide nanoparticle is formed to have a shape selected from the group consisting of: pyramids, discs, plates, and spheres.

20. The method of claim 18, wherein the zinc oxide nanoparticle is formed to have a shape with at least one apex or at least one edge.
